(19) United States
(12) Patent Application Publication
(10) Pub. No.: US 2003/0199578 A1
(43) Pub. Date: Oct. 23, 2003

(54) Naphthalene amides as potassium channel openers

(76) Inventors: Sean C. Turner, Evanston, IL (US); Neil A. Castle, Cary, NC (US); William A. Carroll, Evanston, IL (US); Tammie K. White, Gurnee, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/125,899

(22) Filed: Apr. 19, 2002

(51) Int. Cl. A61K 31/277; A61K 31/165; C07C 255/58; C07C 237/46

(52) U.S. Cl. 514/524; 514/629; 514/657; 558/414; 564/218; 564/428

(57) ABSTRACT

The present invention relates to compounds and their ability to act as potassium channel openers.

### TECHNICAL FIELD

Novel naphthylamide compounds and their derivatives can open potassium channels and are useful for treating urinary incontinence and pain.

### BACKGROUND OF INVENTION

Potassium channels play an important role in regulating cell membrane excitability. For example, when the potassium channels open, changes in the electrical potential across the cell membrane occur and result in a more polarized state. Because there exists a close relationship between potassium channels and cell excitability, many disease states associated with cell excitability can be ameliorated by regulating potassium channel receptors. Such diseases or conditions include asthma, epilepsy, male sexual dysfunction, female sexual dysfunction, pain, bladder overactivity, stroke, diseases associated with decreased skeletal blood flow such as Raynaud's phenomenon and intermittent claudication, eating disorders, functional bowel disorders, neurodegeneration, benign prostatic hyperplasia (BPH), dysmenorrhea, premature labor, alopecia, cardioprotection, coronary artery disease, angina and ischemia.

Potassium channel openers (KCOs) have been shown to act as smooth muscle relaxants, to hyperpolarize bladder cells and consequently relax bladder smooth muscle cells. Because bladder overactivity and urinary incontinence can result from the spontaneous, uncontrolled contractions of the smooth muscle of the bladder, the ability of potassium channel openers to hyperpolarize bladder cells and relax bladder smooth muscle can provide a method to ameliorate or prevent bladder overactivity, pollakiuria, bladder instability, nocturia, bladder hyperreflexia, urinary incontinence, and enuresis.

It is well known that neuronal hyperpolarization can produce analgesic effects. The opening of potassium channels by potassium channel openers and resultant hyperpolarization in the membrane of target neurons is a key mechanism in the effect of opioids. The peripheral antinociceptive effect of morphine results from activation of ATP-sensitive potassium channels, which causes hyperpolarization of peripheral terminals of primary afferents, leading to a decrease in action potential generation. Opening of K_{ATP} channels by potassium channel openers plays an important role in the antinociception mediated by alpha-2 adrenoceptors and mu opioid receptors. KCO's can also potentiate the analgesic action of both morphine and dexmedetomidine via an activation of K_{ATP} channels at the spinal cord level. Thus, potassium channel openers can hyperpolarize neuronal cells and have shown analgesic effects. Potassium channel openers therefore can be used as analgesics in the treatment of various pain states including but not limited to migraine and dyspareunia.

Because of the important role played by potassium channels in Bladder overactivity and pain among many other disease states, there continues to be a need for novel compounds which open potassium channels, relax smooth muscle cells, inhibit bladder contractions and may be useful for treating diseases that can be ameliorated by opening potassium channels.

### SUMMARY OF THE INVENTION

All publications, issued patents and patent applications cited herein are hereby incorporated by reference.

The present invention is directed to compounds of formula (I):

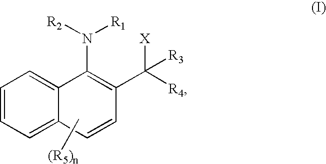

or a pharmaceutically acceptable salt, amide, ester or prodrug thereof wherein,

X is selected from OH, —O-alkyl, —SH, —S-alkyl, —NH₂, —NHR₆, —NR₆R₇;

R₁ and R₂ are independently selected from the group consisting of hydrogen, alkyl, alkylcarbonyl, alkoxycarbonyl, alkylsulfonyl, arylcarbonyl, arylsulfonyl, haloalkylcarbonyl, haloalkylsulfonyl, heterocyclecarbonyl, heterocyclesulfonyl, (NR₈R₉)carbonyl, (NR₈R₉)sulfonyl;

R₃, R₄ is selected from hydrogen, alkyl, aryl, cycloalkyl, haloalkyl, heterocycle;

R₅ is selected from hydrogen, alkenyl, alkenyloxyalkyl, alkoxyalkyl, alkoxycarbonyl, alkoxycarbonylalkyl, alkyl, alkylcarbonyl, alkylcarbonylalkyl, alkylcarbonyloxyalkyl, alkylsulfonylalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, arylalkylthioalkyl, arylsulfonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, heterocyclealkylthioalkyl, hydroxy, hydroxyalkyl, mercaptoalkyl, nitro, NR₁₀R₁₁, (NR₁₀R₁₁)carbonyl, (NR₁₀R₁₁)carbonylalkyl, NR₁₀R₁₁)sulfonyl, (NR₁₀R₁₁)sulfonylalkyl;

R₆, R₇, R₈, R₉ R₁₀ and R₁₁ are each independently selected from hydrogen, alkyl, aryl, arylalkyl, cycloalkyl, cycloalkylalkyl, heterocycle, and heterocyclealkyl; and

n is between 0 and 6.

The present invention is also directed to the use of compounds of formula (I) which are potassium channel openers. Compounds which open potassium channels are useful for the treatment of disorders mediated by potassium channels. In particular one such disorder mediated by potassium channels is bladder overactivity. Accordingly, the present invention provides a method for treating bladder overactivity comprising administering a therapeutically effective amound of a compound of formula (I). Another disorder mediated by potassium channels is pain comprising administering a therapeutically effective amount of a compound of formula (I). Accordingly, the present invention provides a method for treating pain

According to another embodiment, the present invention is directed to a pharmaceutical composition comprising a therapeutically effective amount of a compound of formula (I) in combination with a pharmaceutically acceptable carrier

### DETAILED DESCRIPTION OF THE INVENTION

All publications, issued patents and patent applications cited herein are hereby incorporated by reference.

The present invention is directed to compounds of formula (I):

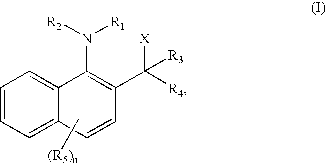

or a pharmaceutically acceptable salt, amide, ester or prodrug thereof wherein,

X is selected from the group consisting of OH, —O-alkyl, —SH, —S-alkyl, —NH₂, —NHR₆, —NR₆R₇;

R₁ and R₂ are independently selected from the group consisting of hydrogen, alkyl, alkylcarbonyl, alkoxycarbonyl, alkylsulfonyl, arylcarbonyl, arylsulfonyl, haloalkylcarbonyl, haloalkylsulfonyl, heterocyclecarbonyl, heterocyclesulfonyl, (NR₈R₉)carbonyl, (NR₈R₉)sulfonyl;

R₃, R₄ is selected from the group consisting of hydrogen, alkyl, aryl, cycloalkyl, haloalkyl, heterocycle;

R₅ is selected from the group consisting of hydrogen, alkenyl, alkenyloxyalkyl, alkoxyalkyl, alkoxycarbonyl, alkoxycarbonylalkyl, alkyl, alkylcarbonyl, alkylcarbonylalkyl, alkylcarbonyloxyalkyl, alkylsulfonylalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, arylalkylthioalkyl, arylsulfonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, heterocyclealkylthioalkyl, hydroxy, hydroxyalkyl, mercaptoalkyl, nitro, NR₁₀R₁₁, (NR₁₀R₁₁)carbonyl, (NR₁₀R₁₁)carbonylalkyl, (NR₁₀R₁₁)sulfonyl, (NR₁₀R₁₁)sulfonylalkyl;

R₆, R₇, R₈, R₉ R₁₀ and R₁₁ are each independently selected from the group consisting of hydrogen, alkyl, aryl, arylalkyl, cycloalkyl, cycloalkylalkyl, heterocycle, and heterocyclealkyl; and

n is between 0 and 6.

According to one embodiment, the present invention is directed to compounds of formula (I), wherein X is hydroxy; and wherein R₁, R₂, R₃, R₄, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl and wherein R₁, R₂, R₅, R₆, R₇, R₈, R₉, R₁₀ , R₁₁ and n are defined in formula (I).

According to one embodiment, the present invention is directed to compounds of formula (I), wherein X is hydroxy; R₃ and R₄ are haloalkyl; R₁ is alkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is alkenylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is alkoxyalkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is cycloalkylalkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is arylalkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is arylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is heterocyclecarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is (NR₆R₇)carbonyl; R₆ is aryl and wherein R₂, R₅, R₇, R₈, R₉, R₁₀, R₁ and n are defined in formula (I).

According to one embodiment, the present invention is directed to compounds of formula (I), wherein X is hydroxy; R₃ and R₄ are haloalkyl; R₁ is alkoxycarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is haloalkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is alkylsulfonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are haloalkyl; R₁ is (NR₈R₉)sulfonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ is haloalkyl; R₄ is aryl; R₁ is alkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is hydroxy; R₃ and R₄ are alkyl; R₁ is alkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is —O-alkyl; R₃ and R₄ are haloalkyl; R is alkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is NH₂; R₃ and R₄ are haloalkyl; R₁ is alkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is NHR₅; R₃ and R₄ are haloalkyl; R₁ is alkylcarbonyl and wherein R₂, R₅, R₆, R₇, R₈, R₉, R₁₀, R₁₁ and n are defined in formula (I).

According to one embodiment, the present invention is directed to compounds of formula (I), wherein X is —SH; R₃ and R₄ are haloalkyl; R₁ is alkylcarbonyl and wherein R₂, R₅, R₆, R₇, R₈, R₉, R₁₀, R₁₁ and n are defined in formula (I).

According to another embodiment, the present invention is directed to a method of treating disorders mediated by potassium channel comprising adminstration of a therapeutically acceptable amount of a compound of formula (I).

According to another embodiment, the present invention is directed to a pharmaceutical composition comprising a therapeutically effective amount of a compound of formula (I) in combination with a pharmaceutically acceptable carrier.

### Definition of Terms

As used throughout this specification and the appended claims, the following terms have the following meanings.

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 1,1-dimethyl-3-butenyl, 4-pentenyl, 5-hexenyl, 2-heptenyl, 2-methyl-1-heptenyl, 3-decenyl and the like.

The term “alkenyloxy,” as used herein, refers to an alkenyl group, as defined herein, appended to the parent molecular moiety through an oxy moiety, as defined herein. Representative examples of alkenyloxy include, but are not limited to, allyloxy, 2-butenyloxy, 3-butenyloxy and the like.

The term “alkenyloxyalkyl,” as used herein, refers to a alkenyloxy group, as defined herein, appended to the parent molecular moiety through an alkyl group, as defined herein. Representative examples of alkenyloxyalkyl include, but are not limited to, (allyloxy)methyl, (2-butenyloxy)methyl and (3-butenyloxy)methyl.

The term “alkoxy,” as used herein, refers to an alkyl group, as defined herein, appended to the parent molecular moiety through an oxy moiety, as defined herein. Representative examples of alkoxy include, but are not limited to, methoxy, ethoxy, propoxy, 2-propoxy, butoxy, tert-butoxy, and the like.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, methoxymethyl, 1,1-dimethyl-3-(methoxy)propyl, and the like.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, tert-butoxycarbonyl, and the like.

The term “alkoxycarbonylalkyl,” as used herein, refers to an alkoxycarbonyl group, as defined herein, appended to the parent molecular moiety through an alkyl group, as defined herein. Representative examples of alkoxycarbonylalkyl include, but are not limited to, methoxycarbonylmethyl, ethoxycarbonylmethyl, tert-butoxycarbonylmethyl, 1,1-dimethyl-2-(methoxycarbonyl)ethyl, and the like.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 1-ethylpropyl, 2,2-dimethylpentyl, 2,3-dimethylpentyl, n-heptyl, n-octyl, n-nonyl, n-decyl, and the like.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, 1-oxopentyl, and the like.

The term “alkylcarbonylalkyl,” as used herein, refers to an alkylcarbonyl group, as defined herein, appended to the parent molecular moiety through an alkyl group, as defined herein. Representative examples of alkylcarbonylalkyl include, but are not limited to, 2-oxopropyl, 1,1-dimethyl-3-oxobutyl, 3-oxobutyl, 3-oxopentyl, and the like.

The term “alkylcarbonyloxy,” as used herein, refers to an alkylcarbonyl group, as defined herein, appended to the parent molecular moiety through an oxy moiety, as defined herein. Representative examples of alkylcarbonyloxy include, but are not limited to, acetyloxy, ethylcarbonyloxy, and the like.

The term “alkylcarbonyloxyalkyl,” as used herein, refers to an alkylcarbonyloxy group, as defined herein, appended to the parent molecular moiety through an alkyl group, as defined herein. Representative examples of alkylcarbonyloxyalkyl include, but are not limited to, acetyloxymethyl, 2-(ethylcarbonyloxy)ethyl, and the like.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl, ethylsulfonyl, and the like.

The term “alkylsulfonylalkyl,” as used herein, refers to an alkylsulfonyl group, as defined herein, appended to the parent molecular moiety through an alkyl group, as defined herein. Representative examples of alkylsulfonylalkyl include, but are not limited to, methylsulfonylmethyl, ethylsulfonylmethyl, and the like.

The term “alkynyl,” as used herein, refers to a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, 1-butynyl, and the like.

The term “aryl,” as used herein, refers to a monocyclic-ring system, or a bicyclic- or a tricyclic- fused ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention can be substituted with 1, 2, or 3 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfonyl, alkynyl, cyano, halo, haloalkyl, haloalkoxy, nitro, phenylalkoxycarbonyl, phenylalkoxycarbonylalkyl, phenylcarbonyloxy, phenylcarbonyloxyalkyl, phenyloxycarbonyl, phenyloxycarbonylalkyl, phenylsulfonyl, NR_{A}R_{B}, (NR_{A}R_{B})alkyl, (NR_{A}R_{B})carbonyl, (NR_{A}R_{B})carbonylalkyl, (NR_{A}R_{B})sulfonyl, (NR_{A}R_{B})sulfonyllalkyl, wherein R_{A} and R_{B} are each independently selected from the group consisting of hydrogen, alkyl and alkylcarbonyl.

The term “arylalkoxy,” as used herein, refers to an aryl group, as defined herein, appended to the parent molecular moiety through an alkoxy group, as defined herein. Representative examples of arylalkoxy include, but are not limited to, 2-phenylethoxy, 3-naphth-2-ylpropoxy, 5-phenylpentyloxy, and the like.

The term “arylalkoxyalkyl,” as used herein, refers to an arylalkoxy group, as defined herein, appended to the parent molecular moiety through an alkyl group, as defined herein. Representative examples of arylalkoxyalkyl include, but are not limited to, 2-phenylethoxymethyl, 2-(3-naphth-2-ylpropoxy)ethyl, 5-phenylpentyloxymethyl, and the like.

The term “arylalkoxycarbonyl,” as used herein, refers to an arylalkoxy group, as defined herein, appended to the parent molecular moiety through a carbonyl group, as defined herein. Representative examples of arylalkoxycarbonyl include, but are not limited to, benzyloxycarbonyl, naphth-2-ylmethyloxycarbonyl, and the like.

The term “arylalkoxycarbonylalkyl,” as used herein, refers to an arylalkoxycarbonyl group, as defined herein, appended to the parent molecular moiety through an alkyl group, as defined herein. Representative examples of arylalkoxycarbonylalkyl include, but are not limited to, benzyloxycarbonylmethyl, 2-(benzyloxycarbonyl)ethyl, 2-(naphth-2-ylmethyloxycarbonyl)ethyl, and the like.

The term “arylalkyl,” as used herein, refers to an aryl group, as defined herein, 1-appended to the parent molecular moiety through an alkyl group, as defined herein. Representative examples of arylalkyl include, but are not limited to, benzyl, 2-phenylethyl, 1,1-dimethyl-2-phenylethyl, 3-phenylpropyl, 2-naphth-2-ylethyl, and the like.

The term “arylalkylthio,” as used herein, refers to an arylalkyl group, as defined herein, appended to the parent molecular moiety through a thio moiety, as defined herein. Representative examples of arylalkylthio include, but are not limited to, 2-phenylethylthio, 3-naphth-2-ylpropylthio, 5-phenylpentylthio, and the like.

The term “arylalkylthioalkyl,” as used herein, refers to an arylalkylthio group, as defined herein, appended to the parent molecular moiety through an alkyl group, as defined herein. Representative examples of arylalkylthioalkyl include, but are not limited to, 2-phenylethylsulfanylmethyl, 3-naphth-2-ylpropylsulfanylmethyl, 2-(5-phenylpentylsulfanyl)ethyl, and the like.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl, naphthoyl, and the like.

The term “arylcarbonylalkyl,” as used herein, refers to an arylcarbonyl group, as defined herein, appended to the parent molecular moiety through an alkyl group, as defined herein. Representative examples of arylcarbonylalkyl include, but are not limited to, 2-oxo-3-phenylpropyl, 1,1-dimethyl-3-oxo-4-phenylbutyl, and the like.

The term “arylcarbonyloxy,” as used herein, refers to an arylcarbonyl group, as defined herein, appended to the parent molecular moiety through an oxy moiety, as defined herein. Representative examples of arylcarbonyloxy include, but are not limited to, benzoyloxy, naphthoyloxy, and the like.

The term “arylcarbonyloxyalkyl,” as used herein, refers to an arylcarbonyloxy group, as defined herein, appended to the parent molecular moiety through an alkyl group, as defined herein. Representative examples of arylcarbonyloxyalkyl include, but are not limited to, benzoyloxymethyl, 2-(benzoyloxy)ethyl, 2-(naphthoyloxy)ethyl, and the like.

The term “aryloxy,” as used herein, refers to an aryl group, as defined herein, appended to the parent molecular moiety through an oxy moiety, as defined herein. Representative examples of aryloxy include, but are not limited to, phenoxy, naphthyloxy, 3-bromophenoxy, 4-chlorophenoxy, 4-methylphenoxy, 3,5-dimethoxyphenoxy, and the like.

The term “aryloxyalkyl,” as used herein, refers to an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl, 3-bromophenoxymethyl, and the like.

The term “aryloxycarbonyl,” as used herein, refers to an aryloxy group, as defined herein, appended to the parent molecular moiety through a carbonyl moiety, as defined herein. Representative examples of aryloxycarbonyl include, but are not limited to, phenoxycarbonyl, naphthyloxycarbonyl, and the like.

The term “aryloxycarbonylalkyl,” as used herein, refers to an aryloxycarbonyl group, as defined herein, appended to the parent molecular moiety through an alkyl group, as defined herein. Representative examples of aryloxycarbonylalkyl include, but are not limited to, phenoxycarbonylmethyl, 2-(phenoxycarbonyl)ethyl, naphthyloxycarbonyl, and the like.

The term “arylsulfonyl,” as used herein, refers to an aryl group, as defined herein, appended to the parent molecular moiety through a sulfonyl group, as defined herein. Representative examples of arylsulfonyl include, but are not limited to, naphthylsulfonyl, phenylsulfonyl, 4-fluorophenylsulfonyl, and the like.

The term “arylsulfonylalkyl,” as used herein, refers to an arylsulfonyl group, as defined herein, appended to the parent molecular moiety through an alkyl group, as defined herein. Representative examples of arylsulfonylalkyl include, but are not limited to, 1,1-dimethyl-3-(phenylsulfonyl)propyl, naphthylsulfonylmethyl, 2-(phenylsulfonyl)ethyl, phenylsulfonylmethyl, 4-fluorophenylsulfonylmethyl, and the like.

The term “carbonyl,” as used herein, refers to a —C(O)— group.

The term “carbonyloxy,” as used herein, refers to a carbonyl group, as defined herein, appended to the parent molecular moiety through an oxy group, as defined herein.

The term “carbonyloxyalkyl,” as used herein, refers to a carbonyloxy group, as defined herein, appended to the parent molecular moiety through an alkyl group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, 3-carboxypropyl, 3-carboxy-1,1-dimethylpropyl and the like.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, 3-cyanopropyl, 3-cyano-1,1-dimethylpropyl, 3-cyano-1,1-diethylpropyl and the like.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3.7}]nonane and tricyclo[3.3.1.1^{3.7}]decane (adamantane).

The cycloalkyl groups of this invention can be substituted with 1, 2, 3, 4, or 5 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfonyl, alkynyl, cyano, halo, haloalkyl, haloalkoxy, nitro, phenylalkoxycarbonyl, phenylalkoxycarbonylalkyl, phenylcarbonyloxy, phenylcarbonyloxyalkyl, phenyloxycarbonyl, phenyloxycarbonylalkyl, phenylsulfonyl, NR_{A}R_{B}, (NR_{A}R_{B})alkyl, (NR_{A}R_{B})carbonyl, (NR_{A}R_{B})carbonylalkyl, (NR_{A}R_{B})sulfonyl, (NR_{A}R_{B})sulfonyllalkyl, wherein R_{A} and R_{B} are described herein.

The term “cycloalkylalkoxy,” as used herein, refers to a cycloalkyl group, as defined herein, appended to the parent molecular moiety through an alkoxy group, as defined herein. Representative examples of cycloalkylalkoxy include, but are not limited to, cyclopropylmethoxy, 2-cyclobutylethoxy, cyclopentylmethoxy, cyclohexylmethoxy, 4-cycloheptylbutoxy, and the like.

The term “cycloalkylalkoxyalkyl,” as used herein, refers to a cycloalkylalkoxy group, as defined herein, appended to the parent molecular moiety through an alkyl group, as defined herein. Representative examples of cycloalkylalkoxyalkyl include, but are not limited to, cyclopropylmethoxymethyl, 2-cyclobutylethoxymethyl, cyclopentylmethoxymethyl, 2-cyclohexylethoxymethyl, 2-(4-cycloheptylbutoxy)ethyl, and the like.

The term “cycloalkylalkyl,” as used herein, refers to a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylethyl and 4-cycloheptylbutyl, and the like.

The term “cycloalkylcarbonyl,” as used herein, refers to a cycloalkyl group, as defined herein, appended to the parent molecular moiety through a carbonyl group, as defined herein. Representative examples of cycloalkylcarbonyl include, but are not limited to, cyclopropylcarbonyl, 2-cyclobutylcarbonyl, cyclohexylcarbonyl, and the like.

The term “cycloalkyloxy,” as used herein, refers to a cycloalkyl group, as defined herein, appended to the parent molecular moiety through an oxy moiety, as defined herein. Representative examples of cycloalkyloxy include, but are not limited to, cyclohexyloxy, cyclopentyloxy, and the like.

The term “cycloalkyloxyalkyl,” as used herein, refers to a cycloalkyloxy group, as defined herein, appended to the parent molecular moiety through an alkyl group, as defined herein. Representative examples of cycloalkyloxyalkyl include, but are not limited to, 4-(cyclohexyloxy)butyl, cyclohexyloxymethyl, and the like.

The term “cycloalkylalkylthio,” as used herein, refers to a cycloalkylalkyl group, as defined herein, appended to the parent molecular moiety through a thio moiety, as defined herein. Representative examples of cycloalkylalkylthio include, but are not limited to, (2-cyclohexylethyl)sulfanyl, cyclohexylmethylsulfanyl, and the like.

The term “cycloalkylalkylthioalkyl,” as used herein, refers to a cycloalkylalkylthio group, as defined herein, appended to the parent molecular moiety through an alkyl group, as defined herein. Representative examples of cycloalkylalkylthioalkyl include, but are not limited to, 2-[(2-cyclohexylethyl)sulfanyl]ethyl, (2-cyclohexylethyl)sulfanylmethyl, and the like.

The term “formyl,” as used herein, refers to a —C(O)H group.

The term “halo” or “halogen,” as used herein, refers to —Cl, —Br, —I or —F.

The term “haloalkenyl,” as used herein, refers to at least one halogen, as defined herein, appended to the parent molecular moiety through an alkenyl group, as defined herein. Representative examples of haloalkenyl include, but are not limited to, 2,2-dichloroethenyl, 2,2-difluoroethenyl, 5-chloropenten-2-yl, and the like.

The term “haloalkyl,” as used herein, refers to at least one halogen, as defined herein, appended to the parent molecular moiety through an alkyl group, as defined herein. Representative examples of haloalkyl include, but are not limited to, chloromethyl, trichloromethyl, 1,1-dichloroethyl, 2-fluoroethyl, trifluoromethyl, 2,2,2-trifluoroethyl, 2,2,2-trifluoro-1-(trifluoromethyl)-1-(methyl)ethyl, pentafluoroethyl, 2-chloro-3-fluoropentyl, and the like.

The term “haloalkynyl,” as used herein, refers to at least one halogen, as defined herein, appended to the parent molecular moiety through an alkyne group, as defined herein. Representative examples of haloalkynyl include, but are not limited to, 1-chlorobut-2-ynyl, 1,1-dichloropent-2-ynyl, 7,7-dichloro-5-methylhept-3-ynyl, and the like.

The term “haloalkylcarbonyl,” as used herein, refers to a haloalkyl group, as defined herein, appended to the parent molecular moiety through a carbonyl group, as defined herein. Representative examples of haloalkylcarbonyl include, but are not limited to, chloromethylcarbonyl, trichloromethylcarbonyl, trifluoromethylcarbonyl, and the like.

The term “haloalkylsulfonyl,” as used herein, refers to a haloalkyl group, as defined herein, appended to the parent molecular moiety through a sulfonyl group, as defined herein. Representative examples of haloalkylsulfonyl include, but are not limited to, chloromethylsulfonyl, trichloromethylsulfonyl, trifluoromethylsulfonyl, and the like.

The term “heterocycle,” as used herein, refers to a monocyclic or a bicyclic ring system. Monocyclic ring systems are exemplified by any 5 or 6 membered ring containing 1, 2, 3, or 4 heteroatoms independently selected from oxygen, nitrogen and sulfur. The 5-membered ring has from 0-2 double bonds and the 6-membered ring has from 0-3 double bonds. Representative examples of monocyclic ring systems include, but are not limited to, azetidine, azepine, aziridine, diazepine, 1,3-dioxolane, dioxane, 1,3-dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydroflran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzotriazole, benzodioxine, 1,3-benzodioxole, cinnoline, indazole, indole, indoline, indolizine, naphthyridine, isobenzofuran, isobenzothiophene, isoindole, isoindoline, 1-isoindolinone, isoquinoline, 1-isoquinolinone, phthalazine, pyranopyridine, quinoline, quinolizine, quinoxaline, quinazoline, tetrahydroisoquinoline, tetrahydroquinoline, and thiopyranopyridine.

The heterocycle groups of this invention can be substituted with 1, 2, or 3 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfonyl, alkynyl, cyano, halo, haloalkyl, haloalkoxy, nitro, phenylalkoxycarbonyl, phenylalkoxycarbonylalkyl, phenylcarbonyloxy, phenylcarbonyloxyalkyl, phenyloxycarbonyl, phenyloxycarbonylalkyl, phenylsulfonyl, NR_{A}R_{B}, (NR_{A}R_{B})alkyl, (NR_{A}R_{B})carbonyl, (NR_{A}R_{B})carbonylalkyl, (NR_{A}R_{B})sulfonyl, (NR_{A}R_{B})sulfonyllalkyl, wherein R_{A} and R_{B} described herein.

The term “heterocyclealkoxy,” as used herein, refers to a heterocycle group, as defined herein, appended to the parent molecular moiety through an alkoxy group, as defined herein. Representative examples of heterocyclealkoxy include, but are not limited to, 2-pyrid-3-ylethoxy, 3-quinolin-3-ylpropoxy, 5-pyrid-4-ylpentyloxy, and the like.

The term “heterocyclealkoxyalkyl,” as used herein, refers to a heterocyclealkoxy group, as defined herein, appended to the parent molecular moiety through an alkyl group, as defined herein. Representative examples of heterocyclealkoxyalkyl include, but are not limited to, 2-pyrid-3-ylethoxymethyl, 2-(3-quinolin-3-ylpropoxy)ethyl, 5-pyrid-4-ylpentyloxymethyl, and the like.

The term “heterocyclealkyl,” as used herein, refers to a heterocycle, as defined herein, appended to the parent molecular moiety through an alkyl group, as defined herein. Representative examples of heterocyclealkyl include, but are not limited to, pyrid-3-ylmethyl, pyrimidin-5-ylmethyl, and the like.

The term “heterocyclealkylthio,” as used herein, refers to a heterocyclealkyl group, as defined herein, appended to the parent molecular moiety through a thio moiety, as defined herein. Representative examples of heterocyclealkylthio include, but are not limited to, 2-pyrid-3-ylethysulfanyl, 3-quinolin-3-ylpropysulfanyl, 5-pyrid-4-ylpentylsulfanyl, and the like.

The term “heterocyclealkylthioalkyl,” as used herein, refers to a heterocyclealkylthio group, as defined herein, appended to the parent molecular moiety through an alkyl group, as defined herein. Representative examples of heterocyclealkylthioalkyl include, but are not limited to, 2-pyrid-3-ylethysulfanylmethyl, 2-(3-quinolin-3-ylpropysulfanyl)ethyl, 5-pyrid-4-ylpentylsulfanylmethyl, and the like.

The term “heterocyclecarbonyl,” as used herein, refers to a heterocycle, as defined herein, appended to the parent molecular moiety through a carbonyl group, as defined herein. Representative examples of heterocyclecarbonyl include, but are not limited to, pyrid-3-ylcarbonyl, quinolin-3-ylcarbonyl, thiophen-2-ylcarbonyl, and the like.

The term “heterocycleoxy,” as used herein, refers to a heterocycle group, as defined herein, appended to the parent molecular moiety through an oxy moiety, as defined herein. Representative examples of heterocycleoxy include, but are not limited to, pyrid-3-yloxy, quinolin-3-yloxy, and the like.

The term “heterocycleoxyalkyl,” as used herein, refers to a heterocycleoxy group, as defined herein, appended to the parent molecular moiety through an alkyl group, as defined herein. Representative examples of heterocycleoxyalkyl include, but are not limited to, pyrid-3-yloxymethyl, 2-quinolin-3-yloxyethyl, and the like.

The term “heterocyclesulfonyl,” as used herein, refers to a heterocycle group, as defined herein, appended to the parent molecular moiety through a sulfonyl group, as defined herein. Representative examples of hetercyclesulfonyl include, but are not limited to pyrid-3-ylsulfonyl, quinolin-3-ylsulfonyl, thiophen-2-ylsulfonyl, and the like.

The term “hydroxy,” as used herein, refers to an —OH group.

The term “hydroxyalkyl,” as used herein, refers to at least one hydroxy groups, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypropyl, 2-ethyl-4-hydroxyheptyl, 2-hydroxy-1,1-dimethylethyl, 3-hydroxy-1,1-dimethylpropyl, and the like.

The term “mercapto,” as used herein, refers to a —SH group.

The term “mercaptoalkyl,” as used herein, refers to a mercapto group, as defined herein, appended to the parent molecular moiety through an alkyl group, as defined herein. Representative examples of mercaptoalkyl include, but are not limited to, 2-sulfanylethyl, 3-sulfanylpropyl, and the like.

The term “NHR₆”, as used herein, refers to R₆, as defined herein, which are appended to the parent molecular moiety through a nitrogen atom. Representative examples of NHR₆ include, but are not limited to, methylamine, dimethylamine, phenylamine, benzylamine, and the like The term “NR₆R₇”, as used herein, refers to R₆ and R₇, as defined herein, which are appended to the parent molecular moiety through a nitrogen atom. Representative examples of NR₆R₇ include, but are not limited to, methylamine, dimethylamine, phenylamine, benzylamine, and the like.

The term “NR₈R₉”, as used herein, refers to R₈ and R₉, as defined herein, which are appended to the parent molecular moiety through a nitrogen atom. Representative examples of NR₈R₉ include, but are not limited to, methylamine, dimethylamine, phenylamine, benzylamine, and the like.

The term “(NR₈R₉)carbonyl”, as used herein, refers to a (NR₈R₉) group as defined herein, appended to the parent molecular moiety through a carbonyl group as defined herein.

Representative examples of (NR₈R₉)carbonyl include, but are not limited to, methylformido, dimethylformido, phenylformido, benzylformido, and the like.

The term “(NR₈R₉)sulfonyl”, as used herein, refers to a (NR₈R₉) group as defined herein, appended to the parent molecular moiety through a sulfonyl group as defined herein.

Representative examples of (NR₈R₉)sulfonyl include, but are not limited to, methylsulfonyl, dimethylsulfonyl, phenylsulfonyl, benzylsulfonyl, and the like.

The term “NR₁₀R₁₁”, as used herein, refers to R₁₀ and R₁₁, as defined herein, which are appended to the parent molecular moiety through a nitrogen atom. Representative examples of NR₁₀R₁₁ include, but are not limited to, methylamine, dimethylamine, phenylamine, benzylamine, and the like.

The term “(NR₁₀R₁₁)alkyl”, as used herein, refers to a (NR₁₀R₁₁) group as defined herein, appended to the parent molecular moiety through a alkyl group as defined herein.

Representative examples of (NR₁₀R₁₁)alkyl include, but are not limited to, N-methyl-N-propylamino, N-dimethyl-N-propylamino, N-phenyl-N-ethylamino, N-benzyl-N-ethylamino, and the like.

The term “(NR₁₀R₁₁)carbonyl”, as used herein, refers to a (NR₁₀R₁₁) group as defined herein, appended to the parent molecular moiety through a carbonyl group as defined herein.

Representative examples of (NR₁₀R₁₁)carbonyl include, but are not limited to, methylformido, dimethylformido, phenylformido, benzylformido, and the like.

The term “(NR₁₀R₁₁)carbonylalkyl”, as used herein, refers to a (NR₁₀R₁₁)carbonyl group as defined herein, appended to the parent molecular moiety through a alkyl group as defined herein. Representative examples of (NR₁₀R₁₁)carbonylalkyl include but are not limited to N-methylpropanamido, N-dimethylpropanamido, N-phenylpropanamido, N-benzylpropanamido, and the like.

The term “(NR₁₀R₁₁)sulfonyl”, as used herein, refers to a (NR₁₀R₁₁) group as defined herein, appended to the parent molecular moiety through a sulfonyl group as defined herein. Representative examples of (NR₁₀R₁₁)sulfonyl include, but are not limited to, methylsulfonyl, dimethylsulfonyl, phenylsulfonyl, benzylsulfonyl, and the like.

The term “(NR₁₀R₁₁)sulfonylalkyl”, as used herein, refers to a (NR₁₀R₁₁)sulfonyl group as defined herein, appended to the parent molecular moiety through an alkyl group as defined herein. Representative examples of (NR₁₀R₁₁)sulfonylalkyl include, but are not limited to, N-methylethanesulfonamido, N-dimethylethanesulfonamido, N-phenylethylsulfonamido, N-benzylethylsulfonamido, and the like.

The term “nitro,” as used herein, refers to a —NO₂ group.

The term “oxo,” as used herein, refers to a (═O) moiety.

The term “oxy,” as used herein, refers to a (—O—) moiety.

The term “oxycarbonyl,” as used herein, refers to an oxy group as defined herein, appended to the parent molecular moiety through a carbonyl group as defined herein.

The term “oxycarbonylalkyl,” as used herein, refers to an oxycarbonyl group as defined herein, appended to the parent molecular moiety through an alkyl group as defined herein.

The term “phenylalkoxycarbonyl,” as used herein, refers to a phenyl group as defined herein, appended to the parent molecular moiety through an alkoxycarbonyl group as defined herein. Representative examples of phenylalkoxycarbonyl include, but are not limited to benzyl formyl, 1-naphthylmethyl formyl, 3-phenylpropyl formyl, and the like.

The term “phenylalkoxycarbonylalkyl,” as used herein, refers to a phenyl group as defined herein, appended to the parent molecular moiety through an alkoxycarbonylalkyl group as defined herein. Representative examples of phenylalkoxycarbonylalkyl include, but are not limited to benzyl propionyl, naphthyl propionyl, 1-phenylethyl propionyl, and the like.

The term “phenylcarbonyloxy,” as used herein, refers to a phenyl group as defined herein, appended to the parent molecular moiety through a carbonyloxy group as defined herein. Representative examples of phenylcarbonyloxy include, but are not limited to benzoyl, 1-naphthoyl, and the like.

The term “phenylcarbonyloxyalkyl,” as used herein, refers to a phenyl group as defined herein, appended to the parent molecular moiety through a carbonyloxyalkyl group as defined herein. Representative examples of phenylcarbonyloxyalkyl include, but are not limited to ethyl benzoyl, propyl benzoate and the like.

The term “phenyloxycarbonyl,” as used herein, refers to a phenyl group as defined herein, appended to the parent molecular moiety through a oxycarbonyl group as defined herein. Representative examples of phenyloxycarbonyl include, but are not limited to phenyl formyl, naphthyl formyl, and the like.

The term “phenyloxycarbonylalkyl,” as used herein, refers to a phenyl group as defined herein, appended to the parent molecular moiety through an oxycarbonylalkyl group as defined herein. Representative examples of phenyloxycarbonylalkyl include, but are not limited to phenyl propionate, naphthyl propionyl, and the like.

The term “phenylsulfonyl,” as used herein, refers to a phenyl group as defined herein, appended to the parent molecular moiety through a sulfonyl group as defined herein. Representative examples of phenylsulfonyl include, but are not limited to phenyl sulfonyl, naphthyl sulfonyl, and the like.

The term “sulfonyl,” as used herein, refers to a —SO₂— group.

The term “tautomer,” as used herein, refers to a proton shift from one atom of a molecule to another atom of the same molecule. The present invention contemplates that particular compounds may exist as tautomers and are contemplated within the scope of the present invention.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Specific compounds of formula (I) include, but are not limited to:

N-[2-(2,2,2-Trifluoro-1-hydroxy-1-trifluoromethyl-ethyl)-naphthalen-1-yl]-acetamide;

N-[6-Hydroxy-2-(2,2,2-trifluoro-1-hydroxy-1-trifluoromethyl-ethyl)-naphthalen-1-yl]-acetamide;

N-[4-Nitro-2-(2,2,2-trifluoro-1-hydroxy-1-trifluoromethyl-ethyl)-naphthalen-1-yl]-acetamide;

8-Acetylamino-7-(2,2,2-trifluoro-1-hydroxy-1-trifluoromethyl-ethyl)-naphthalene-2-sulfonic acid;

5-Acetylamino-6-(2,2,2-trifluoro-1-hydroxy-1-trifluoromethyl-ethyl)-naphthalene-2-sulfonic acid;

4-Acetylamino-3-(2,2,2-trifluoro-1-hydroxy-1-trifluoromethyl-ethyl)-naphthalene-1-sulfonic acid;

N-[5-Hydroxy-2-(2,2,2-trifluoro-1-hydroxy-1-trifluoromethyl-ethyl)-naphthalen-1-yl]-acetamide;

N-[4-Hydroxy-2-(2,2,2-trifluoro-1-hydroxy-1-trifluoromethyl-ethyl)-naphthalen-1-yl]-acetamide;

N-[4-Chloro-2-(2,2,2-trifluoro-1-hydroxy-1-trifluoromethyl-ethyl)-naphthalen-1-yl]-acetamide;

-[4-Cyano-2-(2,2,2-trifluoro-1-hydroxy-1-trifluoromethyl-ethyl)-naphthalen-1-yl]-acetamide;

N-[2-(2,2,2-Trifluoro-1-hydroxy-1-trifluoromethyl-ethyl)-naphthalen-1-yl]-propionamide;

But-2-enoic acid [2-(2,2,2-trifluoro-1-hydroxy-1-trifluoromethyl-ethyl)-naphthalen-1-yl]-amide;

N-[2-(2,2,2-Trifluoro-1-hydroxy-1-trifluoromethyl-ethyl)-naphthalen-1-yl]-butyramide;

N-[2-(2,2,2-Trifluoro-1-hydroxy-1-trifluoromethyl-ethyl)-naphthalen-1-yl]-isobutyramide;

2-Methyl-N-[2-(2,2,2-trifluoro-1-hydroxy-1-trifluoromethyl-ethyl)-naphthalen-1-yl]-butyramide;

3-Methyl-but-2-enoic acid [2-(2,2,2-trifluoro-1-hydroxy-1-trifluoromethyl-ethyl)-naphthalen-1-yl]-amide;

3-Methyl-N-[2-(2,2,2-trifluoro-1-hydroxy-1-trifluoromethyl-ethyl)-naphthalen-1-yl]-butyramide;

Pentanoic acid [2-(2,2,2-trifluoro-1-hydroxy-1-trifluoromethyl-ethyl)-naphthalen-1-yl]-amide;

Pent-4-enoic acid [2-(2,2,2-trifluoro-1-hydroxy-1-trifluoromethyl-ethyl)-naphthalen-1-yl]amide;

Hexanoic acid [2-(2,2,2-trifluoro-1-hydroxy-1-trifluoromethyl-ethyl)-naphthalen-1-yl]-amide;

2-Methyl-pentanoic acid [2-(2,2,2-trifluoro-1-hydroxy-1-trifluoromethyl-ethyl)-naphthalen-1-yl]-amide;

2-Ethyl-N-[2-(2,2,2-trifluoro-1-hydroxy-1-trifluoromethyl-ethyl)-naphthalen-1-yl]-butyramide;

4-Methyl-pentanoic acid [2-(2,2,2-trifluoro-1-hydroxy-1-trifluoromethyl-ethyl)-naphthalen-1-yl]-amide;

3,3-Dimethyl-N-[2-(2,2,2-trifluoro-1-hydroxy-1-trifluoromethyl-ethyl)-naphthalen-1-yl]-butyramide;

2-Methoxy-N-[2-(2,2,2-trifluoro-1-hydroxy-1-trifluoromethyl-ethyl)-naphthalen-1-yl]-acetamide;

Cyclopropanecarboxylic acid [2-(2,2,2-trifluoro-1-hydroxy-1-trifluoromethyl-ethyl)-naphthalen-1-yl]-amide;

Cyclobutanecarboxylic acid [2-(2,2,2-trifluoro-1-hydroxy-1-trifluoromethyl-ethyl)-naphthalen-1-yl]-amide;

Cyclopentanecarboxylic acid [2-(2,2,2-trifluoro-1-hydroxy-1-trifluoromethyl-ethyl)-naphthalen-1-yl]-amide;

2-Cyclopentyl-N-[2-(2,2,2-trifluoro-1-hydroxy-1-trifluoromethyl-ethyl)-naphthalen-1-yl]-acetamide;

Cyclohexanecarboxylic acid [2-(2,2,2-trifluoro-1-hydroxy-1-trifluoromethyl-ethyl)-naphthalen-1-yl]-amide;

3-Phenyl-N-[2-(2,2,2-trifluoro-1-hydroxy-1-trifluoromethyl-ethyl)-naphthalen-1-yl]-propionamide;

N-[2-(2,2,2-Trifluoro-1-hydroxy-1-trifluoromethyl-ethyl)-naphthalen-1-yl]-benzamide;

2-Methyl-N-[2-(2,2,2-trifluoro-1-hydroxy-1-trifluoromethyl-ethyl)-naphthalen-1-yl]-benzamide;

3-Methyl-N-[2-(2,2,2-trifluoro-1-hydroxy-1-trifluoromethyl-ethyl)-naphthalen-1-yl]-benzamide; 4-Methyl-N-[2-(2,2,2-trifluoro-1-hydroxy-1-trifluoromethyl-ethyl)-naphthalen-1-yl]-benzamide;

4-Fluoro-N-[2-(2,2,2-trifluoro-1-hydroxy-1-trifluoromethyl-ethyl)-naphthalen-1-yl]-benzamide;

N-[2-(2,2,2-Trifluoro-1-hydroxy-1-trifluoromethyl-ethyl)-naphthalen-1-yl]-4-trifluoromethyl-benzamide;

4-Bromo-N-[2-(2,2,2-trifluoro-1-hydroxy-1-trifluoromethyl-ethyl)-naphthalen-1-yl]-benzamide;

Biphenyl-4-carboxylic acid [2-(2,2,2-trifluoro-1-hydroxy-1-trifluoromethyl-ethyl)-naphthalen-1-yl]-amide;

N-[2-(2,2,2-Trifluoro-1-hydroxy-1-trifluoromethyl-ethyl)-naphthalen-1-yl]-4-trifluoromethoxy-benzamide;

Isoxazole-5-carboxylic acid [2-(2,2,2-trifluoro-1-hydroxy-1-trifluoromethyl-ethyl)-naphthalen-1-yl]-amide;

N-[2-(2,2,2-Trifluoro-1-hydroxy-1-trifluoromethyl-ethyl)-naphthalen-1-yl]-nicotinamide;

Benzo[b]thiophene-2-carboxylic acid [2-(2,2,2-trifluoro-1-hydroxy-1-trifluoromethyl-ethyl)-naphthalen-1-yl]-amide;

Quinoxaline-2-carboxylic acid [2-(2,2,2-trifluoro-1-hydroxy-1-trifluoromethyl-ethyl)-naphthalen-1-yl]-amide;

3-Fluoro-N-[2-(2,2,2-trifluoro-1-hydroxy-1-trifluoromethyl-ethyl)-naphthalen-1-yl]-benzamide;

3-Bromo-N-[2-(2,2,2-trifluoro-1-hydroxy-1-trifluoromethyl-ethyl)-naphthalen-1-yl]-benzamide;

N-[2-(2,2,2-Trifluoro-1-hydroxy-1-trifluoromethyl-ethyl)-naphthalen-1-yl]-3-trifluoromethyl-benzamide;

3-Methoxy-N-[2-(2,2,2-trifluoro-1-hydroxy-1-trifluoromethyl-ethyl)-naphthalen-1-yl]-benzamide;

3-Nitro-N-[2-(2,2,2-trifluoro-1-hydroxy-1-trifluoromethyl-ethyl)-naphthalen-1-yl]-benzamide;

3-Cyano-N-[2-(2,2,2-trifluoro-1-hydroxy-1-tryfluoromethyl-ethyl)-naphthalen-1-yl]-benzamide;

4-Iodo-N-[2-(2,2,2-trifluoro-1-hydroxy-1-trifluoromethyl-ethyl)-naphthalen-1-yl]-benzamide;

Furan-2-carboxylic acid [2-(2,2,2-trifluoro-1-hydroxy-1-trifluoromethyl-ethyl)-naphthalen-1-yl]-amide;

Thiophene-2-carboxylic acid [2-(2,2,2-trifluoro-1-hydroxy-1-trifluoromethyl-ethyl)-naphthalen-1-yl]-amide;

2,3-Dichloro-N-[2-(2,2,2-trifluoro-1-hydroxy-1-trifluoromethyl-ethyl)-naphthalen-1-yl]-benzainide;

3,4-Dichloro-N-[2-(2,2,2-trifluoro-1-hydroxy-1-trifluoromethyl-ethyl)-naphthalen-1-yl]-benzamide;

3,5-Dichloro-N-[2-(2,2,2-trifluoro-1-hydroxy-1-trifluoromethyl-ethyl)-naphthalen-1-yl]-benzamide;

4-Chloro-N-[2-(2,2,2-trifluoro-1-hydroxy-1-trifluoromethyl-ethyl)-naphthalen-1-yl]-benzamide;

3-Chloro-N-[2-(2,2,2-trifluoro-1-hydroxy-1-trifluoromethyl-ethyl)-naphthalen-1-yl]-benzamide;

2-Chloro-N-[2-(2,2,2-trifluoro-1-hydroxy-1-trifluoromethyl-ethyl)-naphthalen-1-yl]-benzamide;

N-[4-Bromo-2-(2,2,2-trifluoro-1-hydroxy-1-trifluoromethyl-ethyl)-naphthalen-1-yl]-acetamide;

1-(4-Chloro-phenyl)-3-[2-(2,2,2-trifluoro-1-hydroxy-1-trifluoromethyl-ethyl)-naphthalen-1-yl]-urea; and

[2-(2,2,2-Trifluoro-1-hydroxy-1-trifluoromethyl-ethyl)-naphthalen-1-yl]-carbamic acid isopropyl ester

or pharmaceutically acceptable salts, amides, esters or prodrugs thereof.

The following additional compounds, representative of formula (I), may be prepared by one skilled in the art using known synthetic methodology or by using synthetic methodology described in the Schemes and Examples contained herein.

N-(2-(2,2,2-trifluoro-1-hydroxy-1-(trifluoromethyl)ethyl)-1-naphthyl)-3-faramide;

N-(2-(2,2,2-trifluoro-1-hydroxy-1-(trifluoromethyl)ethyl)-1-naphthyl)thiophene-3-carboxamide;

N-(2-(2,2,2-trifluoro-1-hydroxy-1-(trifluoromethyl)ethyl)-1-naphthyl)-1H-imidazole-4-carboxamide

N-(2-(2,2,2-trifluoro-1-hydroxy-1-(trifluoromethyl)ethyl)-1-naphthyl)nicotinamide;

2,2,2-trifluoro-N-(2-(2,2,2-trifluoro-1-hydroxy-1-(trifluoromethyl)ethyl)-1-naphthyl)acetamide;

N-(2-(1-amino-2,2,2-trifluoro-1-(trifluoromethyl)ethyl)-1-naphthyl)acetamide;

N-(2-(2,2,3,3,3-pentafluoro-1-hydroxy-1-(pentafluoroethyl)propyl)-1-naphthyl)acetamide;

N-(2-(2,2,2-trifluoro-1-mercapto-1-(trifluoromethyl)ethyl)-1-naphthyl)acetamide;

N-(2-(1-hydroxy-1-methylethyl)-1-naphthyl)acetamide;

N-(2-(2,2,2-trifluoro-1-hydroxy-1-phenylethyl)-1-naphthyl)acetamide;

N-(2-(2,2,2-trifluoro-1-(methylamino)-1-(trifluoromethyl)ethyl)-1-naphthyl)acetamide;

N-(2-(2,2,2-trifluoro-1-hydroxy-1-(trifluoromethyl)ethyl)-1-naphthyl)methanesulfonamide;

N-(2-(2,2,2-trifluoro-1-hydroxy-1-(trifluoromethyl)ethyl)-1-naphthyl)sulfamide;

N-methyl-N-(2-(2,2,2-trifluoro-1-hydroxy-1-(trifluoromethyl)ethyl)-1-naphthyl)acetamide; and

N-(2-(2,2,2-trifluoro-1-methoxy-1-(trifluoromethyl)ethyl)-1-naphthyl)acetamide.

### Preparation of Compounds of The Invention

The compounds and processes of the present invention will be better understood in connection with the following synthetic schemes and methods which illustrate a means by which the compounds of the invention can be prepared.

The compounds of this invention may be prepared by a variety of synthetic routes. Representative procedures are shown in Schemes 1-7.

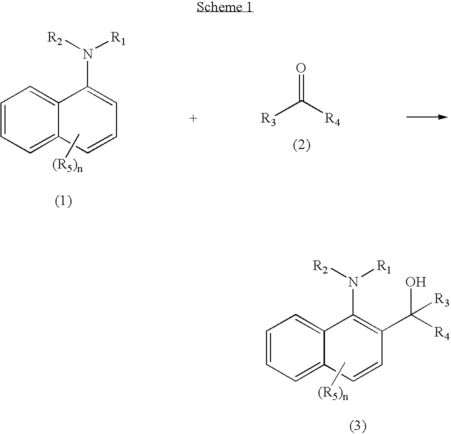

As shown in Scheme 1, naphthylamines of general formula (3), wherein R₁, R₂, R₃, R₄, R₅ and n are as defined in formula (I), may be prepared using the strategy outlined above. Naphthylamines of general formula (1) may be treated with ketones of general formula (2), either by heating neat [Gilbert, E. E., Jones, E. S. and Sibilia, J. P., J. Org. Chem., 1965, 30, 1001-1003], heating in the presence of a catalytic quantity of p-toluenesulfonic acid, or in dichloromethane at room temperature [Chkanikov, N. D., Sviridov, V. D., Zelenin, A. E., Galakhov, M. V., Kolomiets, A. F. and Fokin, A. V., Bull. Acad. Sci. USSR Div. Chem. Sci. (Engl. Transl.), 1990, 39 (2.2), 323-328] to provide a-substituted naphthylamines of general formula (3).

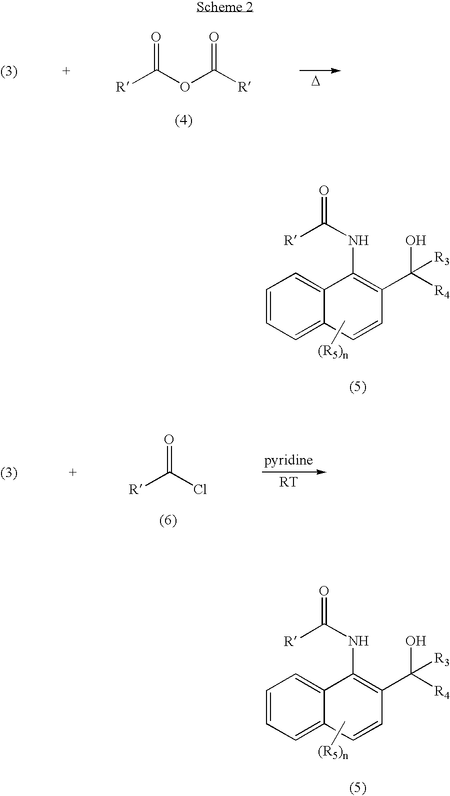

As shown in Scheme 2, naphthylamides of general formula (5), wherein R₃, R₄, R₅ and n are as defined in formula (I), and R is selected from the group consisting of alkyl, aryl, haloalkyl or heterocycle may be prepared using the strategy outlined. Naphthylamines of general formula (3) may be treated with anhydrides of general formula (4), either neat or in the presence of an appropriate carboxylic acid with heating, to provide naphthylamides of general formula (5). Typical solvents used in the reaction include, but are not limited to acetonitrile, and THF, and the like.

Alternatively, naphthylamides of general formula (5), wherein R₃, R₄, R₅ and n are as defined in formula (I), and R′ is selected from the group consisting of alkyl, aryl, haloalkyl or heterocycle may also be prepared by treating naphthylamines of general formula (3) with acid chlorides of general formula (6), in solvents such as pyridine, triethylamine, toluene in the presence of a base such as triethylamine or the reaction may be carried out in solvents such as but not limited to chloroform in the presence of a base such as but not limited to sodium bicarbonate to provide naphthylamides of general formula (5).

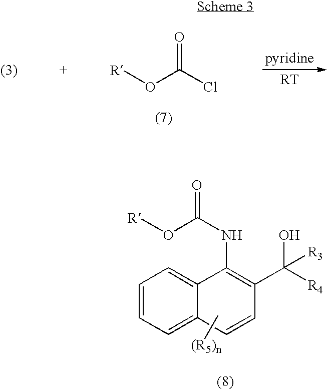

As shown in Scheme 3, naphthylamine carbamate derivatives of general formula (8), wherein R₃, R⁴, R₅ and n are as defined in formula (I), and R′ is selected from the group concsisting of alkyl, aryl, haloalkyl or heterocyclic may be prepared using the above strategy. Naphthylamines of general formula (3) can be reacted with chloroformates of general formula (7), in solvents such as pyridine, or toluene in the presence of pyridine to provide naphthylamine carbamates of general formula (8).

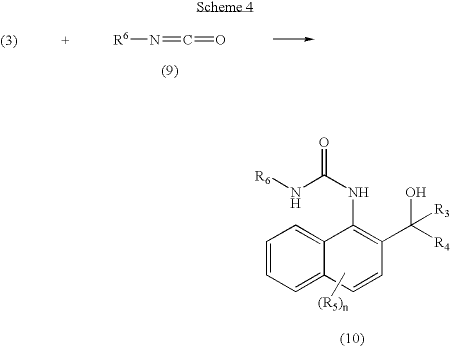

As shown in Scheme 4, naphthylamine ureas of general formula (10), wherein R₃, R₄, R₅, R₆, and n are as defined in formula (1), may be prepared using the above strategy. Naphthylamines of general formula (3) can be reacted with isocyanates of general formula (9), in a solvent such as but not limited to diethyl ether and toluene [Gonda, J. and Barnikol, M., Collect. Czech. Chem. Commun., 1990, 55 (3), 752-760] to provide naphthylamine ureas of general formula (10).

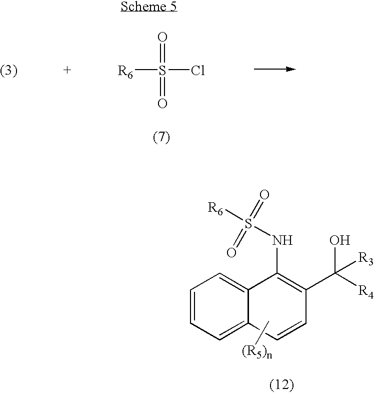

Naphthylamine sulfonamides of general formula (12), wherein R₃, R₄ and R₅ and n are as defined in formula (I), and R₆ is selected from the group consisting of alkyl, aryl and heterocyclic may be prepared using the strategy shown in Scheme 5. Naphthylamines of general formula (3) can be reacted with sulfonyl chlorides of general formula (11), in a solvent such as pyridine [Miura, M., Tsuda, T., Satoh, T., Pivsa-Art, S. and Nomura, M., J. Org. Chem., 1998, 63 (15), 5211-5215] to provide the naphthylamine sulfonamides of general formula (12).

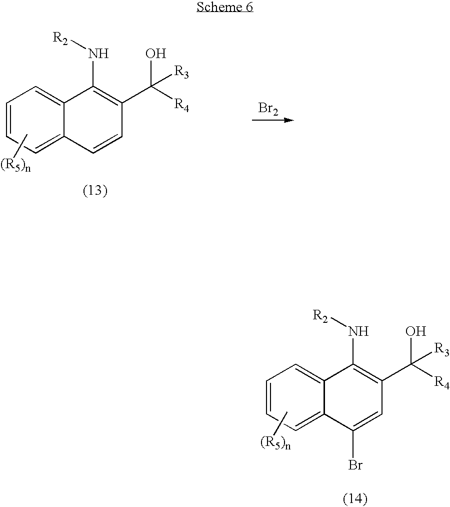

As shown in Scheme 6, naphthylamine derivatives of general formula (14), wherein R₂, R₃, R₄, R₅ and n are as defined in formula I, may be prepared using the strategy outlined above. Naphthylamine derivatives of general formula (13) may be treated with bromine and sodium acetate in a solvent system such as but not limited to acetic acid-tetrahydrofuran to provide 4-substituted naphthylamines derivatives of general formula (14).

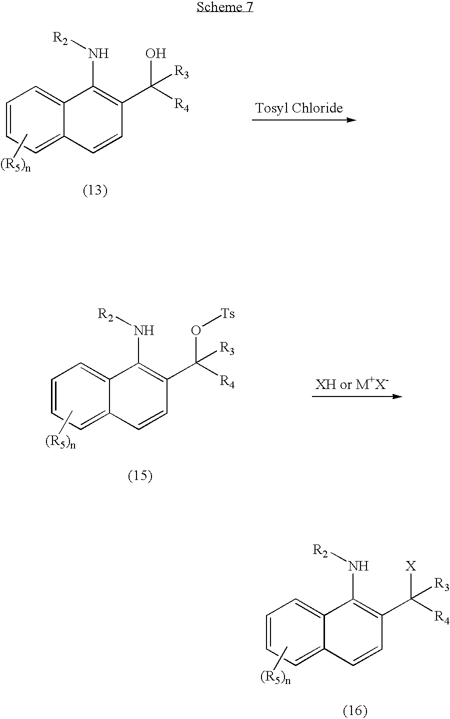

As shown in Scheme 7, naphthylamine derivatives of general formula (16), wherein R₂, R₃, R₄, R₅ and n are as defined in formula I, and X is defined as SH, S-alkyl, NH₂, NHR₆ or NR₆R₇ may be prepared using the strategy outlined above. Naphthylamine derivatives of general formula (13) may be treated with tosyl chloride in a basic solvent system such as but not limited to pyridine to provide 0-tosylated derivatives of general formula (15). Treatment of the intermediate (15) with neutral nucleophiles (for example thiols RSH or amines RNH₂) or metal salts (for example metalated thiols RS-M+or metal amines RNH⁻M⁺) provides naphthylamine derivatives of general formula (16).

The compounds and processes of the present invention will be better understood by reference to the following examples, which are intended as an illustration of and not a limitation upon the scope of the invention. Further, all citations herein are incorporated by reference.

### EXAMPLE 1

### N-[2-(2,2,2-Trifluoro-1-hydroxy-1-trifluoromethyl-ethyl)-naphthalen-1-yl]-acetamide

### Example 1A2-(1-Amino-naphthalen-2-yl)-1,1,1,3,3,3-hexafluoro-propan-2-ol

To a mixture of 1-aminonaphthalene (1.00 g, 7.00 mmol) and p-toluenesulfonic acid (20 mg) was added hexafluoroacetone trihydrate (1.02 mL, 1.61 g, 7.35 mmol). The mixture was stirred in a sealed pressure tube and heated to 120° C. for 16 hours. The reaction was cooled to room temperature and excess hexafluoroacetone trihydrate removed at under reduced pressure to provide the titled compound. The solid was dissolved in diethyl ether (50 mL), filtered through activated charcoal, concentrated in under reduced pressure, and crystallized from 1:1 ethanol-water to give the titled compound (2.16 g, 99%). mp 151-152° C. (lit. 164.5-165.5° C. (E. E. Gilbert, E. S. Jones, J. P. Sibilia, J. Org. Chem., 1965, 1001); ¹H NMR (CDCl₃, 300 MHz) δ7.65 (m, 4H), 7.88 (m, 1H), 7.97 (m, 1H); ¹³C NMR (CDCl₃, 100 MHz) δ93.1, 120.1, 121.3, 125.4, 126.3, 126.9, 127.3, 127.3, 128.8, 130.4, 134.0, 160.1; MS (ESI+) m/z 310 (M+H)⁺; MS (ESI−) m/z 308 (M−H)⁻; Anal. Calcd for C₁₃H₉F₆NO: C, 50.50; H, 2.93; N, 4.53. Found: C, 50.36; H, 2.95; N, 4.43.

### Example 1

### N-[2-(2,2,2-Trifluoro-1-hydroxy-1-trifluoromethyl-ethyl)-naphthalen-1-yl]-acetamide

A mixture of 2-(1-amino-naphthalen-2-yl)-1,1,1,3,3,3-hexafluoro-propan-2-ol (0.75 g, 2.43 mmol) and acetic anhydride (10 mL) was stirred in a sealed pressure tube heated to 120° C. for 2 hours. The mixture was cooled to room temperature and excess acetic anhydride removed under reduced pressure. The residue was purified using flash chromatography (silica gel, gradient elution of 100% hexane to 3:1 ethyl acetate-hexane) to provided the titled compound (520 mg, 61%). mp 155-156° C.; ¹H NMR (CDCl₃, 300 MHz) rotamers δ1.68, 2.28, 2.38 (s, 3H, CH₃), 5.61 (br s, 1H), 7.56-8.05 (m, 6H); MS (ESI+) m/z 352 (M+H)⁺; MS (ESI−) m/z 350 (M−H)⁻; Anal. Calcd for C₁₅H₁₁F₆NO₂: C, 51.29; H, 3.16; N, 3.99. Found: C, 51.50; H, 3.13; N, 3.87.

### Example 2

### N-[6-Hydroxy-2-(2,2,2-trifluoro-1-hydroxy-1-trifluoromethyl-ethyl)-naphthalen-1-yl]-acetamide

### Example 2A

### 5-Amino-6-(2,2,2-trifluoro-1-hydroxy-1-trifluoromethyl-ethyl)-naphthalen-2-ol

5-Amino-2-naphthol was substituted for 1-aminonaphthalene and processed as described in Example 1A to provide the titled compound as a brown solid. The residue was purified using flash chromatography (silica gel, gradient elution of 100% hexane to 3:1 ethyl acetate-hexane) to provide the titled compound (3.59 g, 88%). mp 160-161° C.; ¹H NMR (CD₃OD, 300 MHz) δ7.02 (m, 4H), 7.27 (m, 1H), 7.97 (d, 1H); MS (ESI+) m/z 326 (M+H)⁺; MS (ESI−) m/z 324 (M−H)⁻; Anal. Calcd for C₁₃H₉F₆NO₂: C, 48.01; H, 2.79; N, 4.31. Found: C, 47.98; H, 2.60; N, 4.21.

### Example 2

### N-[6-Hydroxy-2-(2,2,2-trifluoro-1-hydroxy-1-trifluoromethyl-ethyl)-naphthalen-1-yl]-acetamide

The product from Example 2A was processed as described in Example 1. The product was purified using reverse-phase chromatography provided the titled compound (240 mg, 10%). ¹H NMR (D₆-DMSO, 300 MHz) δ2.14 (s, 3H), 7.16 (m, 2H), 7.68 (m, 3H), 8.71 (s, 1H), 9.28 (s, 1H), 10.04 (s, 1H); MS (ESI+) m/z 368 (M+H)⁺; MS (ESI−) m/z 366 (M−H)⁻; Anal. Calcd for C₁₅H₁₁F₆NO₃+0.15CH₂Cl₂: C, 47.89; H, 3.00; N, 3.69. Found: C, 47.94; H, 2.67; N, 3.52.

### Example 3

### N-[4-Nitro-2-(2,2,2-trifluoro-1-hydroxy-1-trifluoromethyl-ethyl)-naphthalen-1-yl]-acetamide

### Example 3A

### 2-(1-Amino-4-nitro-naphthalen-2-yl)-1,1,1,3,3,3-hexafluoro-propan-2-ol

1-Amino-4-nitronaphthalene was substituted for 1-aminonaphthalene and processed as described in Example 1A to provide the titled compound as a yellow solid. The solid was crystallized from toluene, dissolved in dichloromethane and filtered. Concentration of the filtrate provided the titled compound (61%).

¹H NMR (CD₃OD, 300 MHz) δ7.67 (t, 1H), 7.86 (t, 1H), 7.96 (s, 1H), 8.47 (d, 1H), 8.60 (s, 1H), 8.84 (d, 1H), 9.93 (s, 1H); MS (ESI+) m/z 355 (M+H)⁺; MS (EST−) m/z 353 (M−H)⁻; Anal. Calcd for C₁₃H₈F₆N₂O₃: C, 44.08; H, 2.28; N, 7.91. Found: C, 43.81; H, 2.28; N, 7.82.

### Example 3

### N-[4-Nitro-2-(2,2,2-trifluoro-1-hydroxy-1-trifluoromethyl-ethyl)-naphthalen-1-yl]-acetamide

The product from Example 3A was processed as described in Example 1 with a change in the reaction time from 2 hours to 60 hours. Purification using reverse-phase chromatography provided the titled compound (16 mg, 16%).

¹H NMR (CD₃OD, 300 MHz) δ2.30 (s, 3H), 7.77 (t, 1H), 7.86 (t, 1H), 8.13 (d, 1H), 8.50 (d, 1H), 8.58 (s, 1H); MS (ESI+) m/z 397 (M+H, 80); MS (ESI−) m/z 395 (M−H, 100); HRMS 397.0627 (397.0623 calcd for C₁₅H₁₁N₂O₄F₆).

### Example 4

### 8-Acetylamino-7-(2,2,2-trifluoro-1-hydroxy-1-trifluoromethyl-ethyl)-naphthalene-2-sulfonic acid

### Example 4AB

### 8-Amino-7-(2,2,2-trifluoro-1-hydroxy-1-trifluoromethyl-ethyl -naphthalene-2-sulfonic acid

To a mixture of 8-amino-2-naphthalenesulfonic acid (1.56 g, 7.00 mmol) and p-toluenesulfonic acid (20 mg) was added diisopropylethylamine (0.90 g, 7.00 mmol) and hexafluoroacetone trihydrate (1.02 mL, 1.61 g, 7.35 mmol). The mixture was stirred in a sealed pressure tube and heated to 120° C. for 16 hours. The reaction was cooled to room temperature and excess reactants removed at under reduced pressure to provide the titled compound. Reverse-phase chromatography provided the titled compound (240 mg, 10%). ¹H NMR (d₆-DMSO, 300 MHz) δ7.22 (m, 2H), 7.72 (m, 2H), 8.42 (s, 1H); MS (ESI+) m/z 388 (M−H, 100); Anal. Calcd for C₁₃H₉F₆NO₄S: C, 40.11; H, 2.33; N, 3.60. Found: C, 39.91; H, 2.21; N, 3.58.

### Example 4

### 8-Acetylamino-7-(2,2,2-trifluoro-1-hydroxy-1-trifluoromethyl-ethyl)-naphthalene-2-sulfonic acid

The product from Example 4A was processed as described in Example 1 with a change in the reaction time from 2 hours to 16 hours. Purification using reverse-phase chromatography provided the titled compound (%). ¹H NMR (CD₃OD, 300 MHz) δ rotamers 2.38 (s, 3H), 7.60 (d, 1H), 7.95 (2×d, 1H), 8.00 (s, 1H), 8.05 (2×d, 1H), 9.16 (s, 1H); MS (ESI+) m/z 432 (M+H, 12); MS (ESI−) m/z 430 (M−H, 100); HRMS 432.0333 (432.0340 calcd for C₁₅H₁₂NO₅F₆S).

### Example 5

### 5-Acetylamino-6-(2,2,2-trifluoro-1-hydroxy-1-trifluoromethyl-ethyl -naphthalene-2-sulfonic acid

### Example 5A

### 5-Amino-6-(2.2,2-trifluoro-1-hydroxy-1-trifluoromethyl-ethyl)-naphthalene-2-sulfonic acid

8-Amino-2-naphthalenesulfonic acid was processed as described in Example 4A to provide the titled compound (255 mg, 10%). ¹H NMR (d₆-DMSO, 300 MHz) δ7.24 (m, 2H), 7.64 (m, 1H), 7.98 (s, 1H), 8.18 (m, 1H); MS (ESI−) m/z 388 (M−H); Anal. Calcd for C₁₃H₉F₆NO₄S+0.3H₂O: C, 39.56; H, 2.45; N, 3.55. Found: C, 39.29; H, 2.19; N, 3.56.

### Example 5

### 5-Acetylamino-6-(2,2,2-trifluoro-1-hydroxy-1-trifluoromethyl-ethyl)-naphthalene-2-sulfonic acid

The product from Example 5A was processed as described in Example 1 with a change in the reaction time from 2 hours to 16 hours. Purification by reverse-phase chromatography provided the titled compound (%). ¹H NMR (CD₃OD, 300 MHz) δ2.39 (s, 3H), 7.60 (d, 1H), 7.95 (2×d, 1H), 8.00 (m, 2H), 8.38 (s, 1H), 8.73 (d, 1H); MS (ESI+) m/z 432 (M+H, 14); MS (ESI−) m/z 430 (M−H, 75); HRMS 432.0340 (432.0340 calcd for C₁₅H₁₂NO₅F₆S).

### Example 6

### 4-Acetylamino-3-(2,2,2-trifluoro-1-hydroxy-1-trifluoromethyl-ethyl)-naphthalene-1-sulfonic acid

### Example 6A

### 4-Amino-3-(2,2,2-trifluoro-1-hydroxy-1-trifluoromethyl-ethyl)-naphthalene-1-sulfonic acid

4-Amino-naphthalene-1-sulfonic acid was substituted for 1-aminonaphthalene and processed as described in Example 4A to provide the titled compound. ¹H NMR (d₆-DMSO, 300 MHz) δ7.48 (m, 2H), 7.82 (m, 1H), 7.96 (s, 1H), 8.18 (m, 1H); MS (ESI+) m/z 390 (M−H, 30); MS (ESI−) m/z 388 (M−H, 100); HRMS 389.0139 (389.0156 calcd for C₁₃H₉NO₄F₆S).

### Example 6

### 4-Acetylamino-3-(2,2,2-trifluoro-1-hydroxy-1-trifluoromethyl-ethyl -naphthalene-1-sulfonic acid

The product from Example 6A was processed as described in Example 1 with a change in the reaction time from 2 hours to 16 hours. Purification by reverse-phase chromatography provided the titled compound. ¹H NMR (CD₃OD, 300 MHz) δ rotamers 2.24, 2.40 (2×s, 3H), 7.83 (m, 2H), 8.05-8.25 (m, 1.5H), 8.78-8.90 (m, 1.5H); MS (ESI+) m/z 432 (M+H, 10); MS (ESI−) m/z 430 (M−H, 100); HRMS 432.0333 (432.0340 calcd for C₁₅H₁₂NO₅F₆S).

### Example 7

### N-[5-Hydroxy-2-(2,2,2-trifluoro-1-hydroxy-1-trifluoromethyl-ethyl)-naphthalen-1-yl]-acetamide

### Example 7A

### 5-Amino-6-(2,2,2-trifluoro-1-hydroxy-1-trifluoromethyl-ethyl)-naphthalen-1-ol

5-Amino-1-naphthol was substituted for 1-aminonaphthalene and processed as described in Example 1A to provide the titled compound as a brown solid. Purification by flash chromatography (silica gel, gradient elution of 100% hexane to 3:1 ethyl acetate-hexane) provided the titled compound (472 mg, 21%). ¹H NMR (d₆-DMSO, 300 MHz) δ6.90 (d, 1H), 7.20 (d, 1H), 7.27 (t, 1H), 7.44 (d, 1H), 7.59 (d, 1H); MS (ESI+) m/z 326 (M+H)⁺; MS (ESI−) m/z 324 (M−H)−; Anal. Calcd for C₁₃H₉F₆NO₂: C, 48.01; H, 2.79; N, 4.31. Found: C, 47.94; H, 2.69; N, 4.28.

### Example 7

### N-[5-Hydroxy-2-(2,2,2-trifluoro-1-hydroxy-1-trifluoromethyl-ethyl -naphthalen-1-yl]-acetamide

The product from Example 7A was processed as described in Example 1. Reverse-phase chromatography provided the titled compound (26 mg, 12%). ¹H NMR (CD₃OD, 300 MHz) δ2.22 (s, 3H), 6.88 (d, 1H), 7.39 (m, 2H), 7.70 (d, 1H), 8.23 (d, 1H); MS (ESI+) m/z 368 (M+H)⁺; MS (ESI−) m/z 366 (M−H)⁻; Anal. Calcd for C₁₅H₁₁F₆NO₃+0.15 CH₂Cl₂: C, 47.89; H, 3.00; N, 3.69. Found: C, 47.94; H, 2.67; N, 3.52.

### Example 8

### N-[4-Hydroxy-2-(2,2,2-trifluoro-1-hydroxy-1-trifluoromethyl-ethyl)-naphthalen-1-yl]-acetamide

### Example 8A

### 4-Amino-3-(2,2,2-trifluoro-1-hydroxy-1-trifluoromethyl-ethyl)-naphthalen-1-ol

4-Amino-naphthalen-1-ol was substituted for 1-aminonaphthalene and processed as described in Example 1A to provide the titled compound (8%). ¹H NMR (d₆-DMSO, 300 MHz) δ7.26 (s, 1H), 7.81 (m, 2H), 7.92 (d, 1H), 8.21 (d, 1H); MS (ESI+) m/z 326 (M+H, 100); MS (ESI−) m/z 324 (M-H, 100); HRMS 325.0527 (325.0537 calcd for C₁₃H₉NO₂F₆).

### Example 8

### N-[4-Hydroxy-2-(2,2,2-trifluoro-1-hydroxy-1-trifluoromethyl-ethyl)-naphthalen-1-yl]-acetamide

The product from Example 8A was processed as described in Example 1. Reverse-phase chromatography provided the titled compound (26 mg, 28%). ¹H NMR (CD₃OD, 300 MHz) δ2.24 (s, 3H), 7.62 (m, 2H), 7.80 (d, 1H), 7.93 (m, 1H), 8.02 (m, 1H); MS (ESI+)m/z 368 (M+H)⁺; MS (ESI−) m/z 366 (M−H)⁻;

### Example 9

### N-[4-Chloro-2-(2,2,2-trifluoro-1-hydroxy-1-trifluoromethyl-ethyl)-naphthalen-1-yl]-acetamide

### Example 9A

### 2-(1-Amino-4-chloro-naphthalen-2-yl)-1,1,1,3,3,3-hexafluoro-propan-2-ol

1-Amino-4-chloronaphthalene was substituted for 1-aminonaphthalene and processed as described in Example IA to provide the titled compound as a purple solid. The solid was recrystallized from 4:1 water-methanol to provide the titled compound (1.47 g, 61%). ¹H NMR (d₆-DMSO, 300 MHz) 8 7.61 (t, 1H), 7.74 (t, 1H), 8.02 (d, 1H), 8.33 (d, 1H), 9.70 (s, 1H); MS (ESI+) m/z 344 (M+H)⁺; MS (ESI−) m/z 342 (M−H)⁻; HRMS 343.0196 (343.0199 calcd for C₁₃H₈ClNOF₆).

### Example 9

### N-[4-Chloro-2-(2,2,2-trifluoro-1-hydroxy-1-trifluoromethyl-ethyl)-naphthalen-1-yl]-acetamide

The product from Example 9A (50 mg, 0.14 mmol), acetic anhydride (50 mg) and acetic acid (0.2 mL) were stirred in a sealed pressure tube and heated to 120° C. for 2 h. The reaction was cooled to room temperature and solvent removed at pump. Reverse-phase chromatography provided the titled compound (14 mg, 27%). mp 161-162° C.; ¹H NMR (d6-DMSO, 300 MHz) δ2.48 (s, 3H), 6.65 (s, 1H), 7.32 (s, 1H), 7.63 (dt, 1H), 7.73 (dt, 1H), 8.05 (dd, 1H), 8.32 (d, 1H), 9.72 (s, 1H); MS (ESI+) m/z 386 (M+H)⁺; Anal. Calcd for C₁₅H₁₀ClF₆NO₂+0.3TFA: C, 44.62; H, 2.47; N, 3.34. Found: C, 44.57; H, 2.41; N, 3.65.

### Example 10

### N-[4-Cyano-2-(2,2,2-trifluoro-1-hydroxy-1-trifluoromethyl-ethyl)-naphthalen-1-yl]-acetamide

### Example 10A

4-Amino-3-(2,2,2-trifluoro-1-hydroxy-1-trifluoromethyl-1-ethyl)-naphthalene-1-carbonitrile

1-Amino-4-cyanonaphthalene was substituted for 1-aminonaphthalene and processed as described in Example 1A to provide the titled compound (0.59 g, 25%). ¹H NMR (d₆-DMSO, 300 MHz) δ7.52 (s, 1H), 7.65 (t, 1H), 7.80 (t, 1H), 7.94 (d, 1H), 8.43 (d, 1H, 9.76 (s, 1H); MS (ESI+) m/z 335 (M+H)⁺; MS (ESI−) m/z 333 (M−H)⁻; Anal. Calcd for C₁₄H₈F₆N₂O: C, 50.31; H, 2.41; N, 8.38. Found: C, 50.38; H, 2.51; N, 8.42.

### Example 10

### N- [4-Cyano-2-(2,2,2-trifluoro-1-hydroxy-1-trifluoromethyl-ethyl)-naphthalen-1-yl]-acetamide

The product from Example 10A was processed as described in Example 1. Reverse-phase chromatography provided the titled compound (41 mg, 17%). ¹H NMR (d₆-DMSO, 300 MHz) δ7.72 (t, 1H), 7.85 (t, 1H), 8.06 (d, 1H), 8.23 (m, 2H); MS (ESI+) m/z 377 (M+H)⁺; MS (ESI−) m/z 375 (M−H)⁻; HRMS 376.0652 (376.0646 calcd for C₆H₁₀N₂O₂F₆).

### Example 11

### N-[2-(2,2,2-Trifluoro-1-hydroxy-1-trifluoromethyl-ethyl)-naphthalen-1-yl]-propionamide

2-(1-Amino-naphthalen-2-yl)-1,1,1,3,3,3-hexafluoro-propan-2-ol and propanoic anhydride were processed as described in Example 1. Reverse-phase chromatography provided the titled compound (170 mg, 28%). m.p. 118-119° C; ¹H NMR (CDCl₃, 300 MHz) δ1.33 (t, 3H), 2.62 (t, 2H), 7.49 (m, 1H), 7.62 (m, 2H), 7.78 (m, 1H), 7.84 (m, 1H), 8.74 (m, 1H); MS (ESI+) m/z 366 (M+H)⁺; MS (ESI−) m/z 364 (M−H)⁻; Anal. Calcd for C₁₆H₁₃F₆NO₂: C, 52.61; H, 3.59; N, 3.83. Found: C, 52.95; H, 3.27; N, 3.53.

### Example 12

### But-2-enoic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and but-2-enoic anhydride were processed as described in Example 1. Reverse-phase chromatography provided the titled compound (17 mg, 7%). m.p. 216-217° C.; ¹H NMR (CDCl₃, 300 MHz) δ1.98 (m, 3H), 5.64 (s, 1H), 6.21 (d, 1H), 7.10 (m, 1H), 7.60 (m, 3H), 7.72 (m, 1H), 7.88 (m, 3H); MS (ESI+) m/z 378 (M+H)⁺; MS (ESI−) m/z 376 (M−H−)⁻; HRMS 377.0845 (377.0850 calcd for C₁₇H₁₃NO₂F₆).

### Example 13

### N-[2-(2,2,2-Trifluoro-1-hydroxy-1-trifluoromethyl-ethyl -naphthalen-1-yl]-butyramide

A mixture of 2-(1-amino-naphthalen-2-yl)-1,1,1,3,3,3-hexafluoro-propan-2-ol(200 mg, 0.65 mmol) in dry pyridine (2 mL) was added to a stirred solution of propionyl chloride (138 mg, 1.29 mmol) in dry pyridine (2 mL) and stirred at room temperature for 4 h. The pyridine was removed at pump and the residue purified by reverse-phase chromatography to provide the titled compound (56 mg, 23%). m.p. 189-190° C.; ¹H NMR (CDCl₃, 300 MHz) δ1.06 (t, 3H), 1.85 (m, 2H), 2.59 (t, 2H), 7.38 (d, 1H), 7.51 (m, 2H), 7.69 (d, 1H), 7.73 (m, 1H), 8.61 (m, 1H); MS (ESI+) m/z 380 (M+H)⁺; MS (ESI−) m/z 378 (M−H)⁻; Anal. Calcd for C₁₇H₁₅F₆NO₂: C, 53.83; H, 3.99; N, 3.69. Found: C, 54.19; H, 4.01; N,

### Example 14

### N-[2-(2,2,2-Trifluoro-1-hydroxy-1-trifluoromethyl-ethyl)-nahthalen-1-yl]-isobutyramide

2-(1-Amino-naphthalen-2-yl)-1,1,1,3,3,3-hexafluoro-propan-2-ol and isobutyryl chloride were processed as described in Example 13 to provide the titled compound (29 mg, 12%). ¹H NMR (CDCl₃, 300 MHz) δ1.37 (d, 6H), 2.82 (m, 1H), 7.43 (d, 1H), 7.60 (m, 2H), 7.80 (m, 2H), 8.73 (m, 1H); MS (ESI+) m/z 380 (M+H)⁺; MS (ESI−) m/z 378 (M−H)⁻; Anal. Calcd for C₁₇H₁₅F₆NO₂: C, 53.83; H, 3.99; N, 3.69. Found: C, 53.49; H, 3.88; N, 3.61.

### Example 15

### 2-Methyl-N-[2-(2,2,2-trifluoro-1-hydroxy-1-trifluoromethyl-ethyl)-naphthalen-1-yl]-butyramide

2-(1-Amino-naphthalen-2-yl)-1,1,1,3,3,3-hexafluoro-propan-2-ol and 2-methylbutyryl chloride were processed as described in Example 13 to provide the titled compound (61 mg, 24%). m.p. 167-168° C.; ¹H NMR (CDCl₃, 300 MHz) δ1.02 (t, 3H), 1.37 (d, 3H), 1.64 (m, 1H), 1.90 (m, 1H), 2.61 (m, 1H), 7.43 (d, 1H), 7.60 (m, 2H), 7.80 (m, 2H), 8.73 (m, 1H); MS (ESI+) m/z 394 (M+H)⁺; MS (ESI−) m/z 392 (M−H)⁻; Anal. Calcd for C₁₈H₁₇F₆NO₂+0.2 TFA: C, 53.11; H. 4.17; N, 3.37. Found: C, 52.99; H. 3.88; N, 3.47.

### Example 16

### 3-Methyl-but-2-enoic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and 3-methyl-but-2-enoyl chloride were processed as described in Example 13 to provide the titled compound (37.4 mg, 15%). m.p. 135-137° C.; ¹H NMR (CDCl₃, 300 MHz) δ2.01 (s, 3H), 2.23 (s, 3H), 5.25 (m, 1H), 6.07 (m, 1H), 7.73 (m, 7H); MS (ESI+) m/z 391 (M+H)⁺; MS (ESI−) m/z 389 (M−H)⁻; Anal. Calcd for C₁₈H₁₅F₆NO₂: C, 55.25; H, 3.86; N, 3.58. Found: C, 54.88; H, 3.69; N, 3.63.

### Example 17

### 3-Methyl-N-[2-(2.2,2-trifluoro-1-hydroxy-1-trifluoromethyl-ethyl)-naphthalen-1-yl]-butyramide

2-(1-Amino-naphthalen-2-yl)-1,1,1,3,3,3-hexafluoro-propan-2-ol and 3-methyl-butyryl chloride were processed as described in Example 13 to provide the titled compound (18 mg, 10%). ¹H NMR (CDCl₃, 300 MHz) δ1.08 (d, 6H), 2.31 (m, 1H), 2.48 (d, 1H), 7.49 (m, 1H), 7.62 (m, 2H), 7.77 (m, 1H), 7.84 (m, 1H), 8.72 (m, 1H); MS (ESI+) m/z 394 (M+H)⁺; MS (ESI−) m/z 392 (M−H)⁻; HRMS 394.1234 (394.1242 calcd for C₁₈H₁₈NO₂F₆).

### Example 18

### Pentanoic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and pentanoyl chloride were processed as described in Example 13 to provide the titled compound (25 mg, 10%). ¹H NMR (CDCl₃, 300 MHz) δ1.00 (t, 3H), 1.49 (sextet, 2H), 1.82 (quintet, 2H), 2.61 (t, 2H), 7.49 (m, 1H), 7.62 (m, 2H), 7.77 (m, 1H), 7.84 (m, 1H), 8.72 (m, 1H); MS (ESI+) m/z 394 (M+H)⁺; MS (ESI−) m/z 392 (M−H)⁻; HRMS 394.1242 (394.1242 calcd for C₁₈H₁₈NO₂F₆).

### Example 19

### Pent-4-enoic acid [2-(2,2,2-trifluoro-1-hydroxy-1-trifluoromethyl-ethyl)-naphthalen-1-yl]amide

2-(1-Amino-naphthalen-2-yl)-1,1,1,3,3,3-hexafluoro-propan-2-ol and pent-4-enoyl chloride were processed as described in Example 13 to provide the titled compound (44 mg, 23%). ¹H NMR (CDCl₃, 300 MHz) δ2.60 (m, 2H), 2.66 (m, 2H), 5.02-5.20 (m, 2H), 5.94 (m, 1H), 7.45-7.90 (m, 5H), 8.72 (m, 1H); MS (ESI+) m/z 392 (M+H)⁺; MS (ESI−) m/z 390 (M−H)⁻; HRMS 392.1072 (302.1085 calcd for C₁₈H₁₆NO₂F₆).

### Example 20

### Hexanoic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and hexanoyl chloride were processed as described in Example 13 to provide the titled compound (27 mg, 25 13%). ¹H NMR (CDCl₃, 300 MHz) δ0.88 (t, 3H), 1.25-1.50 (m, 4H), 1.82 (m, 2H), 2.60 (t, 2H), 7.47 (m, 1H), 7.62 (m, 2H), 7.72-7.88 (m, 2H), 8.68 (m, 1H); MS (ESI+) m/z 408 (M+H)⁺; MS (ESI−) m/z 406 (M−H)⁻; HRMS 408.1388 (408.1398 calcd for C₁₉H₂₀NO₂F₆).

### Example 21

### 2-Methyl-pentanoic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and 2-methylpentanoyl chloride were processed as described in Example 13 to provide the titled compound (29 mg, 11%). ¹H NMR (CDCl₃, 300 MHz) δ0.98 (t, 3H), 1.37 (d, 3H), 1.43 (m, 2H), 1.60 (m, 2H), 1.85 (m, 1H), 2.75 (m, 1H), 7.46 (d, 1H), 7.60 (m, 2H), 7.78 (d, 1H), 7.81 (m, 1H), 8.73 (m, 1H); MS (ESI+) m/z 408 (M+H)⁺; MS (ESI−) m/z 406 (M−H)⁻; Anal. Calcd for C₁₉H₁₉F₆NO₂: C, 56.02; H, 4.70; N, 3.44. Found: C, 55.95; H, 4.61; N, 3.23.

### Example 22

### 2-Ethyl-N-[2-(2,2,2-trifluoro-1-hydroxy-1-trifluoromethyl-ethyl)-naphthalen-1-yl]-butyramide

2-(1-Amino-naphthalen-2-yl)-1,1,1,3,3,3-hexafluoro-propan-2-ol and 2-ethylbutyryl chloride were processed as described in Example 13 to provide the titled compound (26 mg, 10%). ¹H NMR (CDCl₃, 300 MHz) δ1.04 (t, 6H), 1.70 (m, 3H), 1.84 (m, 3H), 2.40 (m, 1H), 5.81 (br s, 1H), 7.60 (m, 2H), 7.71 (m, 2H), 7.89 (m, 2H); MS (ESI+) m/z 408 (M+H)⁺; MS (ESI−) m/z 406 (M−H)⁻; Anal. Calcd for C₁₉H₁₉F₆NO₂: C, 56.02; H, 4.70; N, 3.44. Found: C, 55.96; H, 4.62; N, 3.32.

### Example 23

### 4-Methyl-pentanoic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and 4-methyl-pentanoyl chloride were processed as described in Example 13 to provide the titled compound (20 mg, 10%). mp 62-63° C. ¹H NMR (CDCl₃, 300 MHz) δ0.92 (m, 6H), 1.58 (m, 1H), 1.72 (m, 2H), 2.60 (m, 2H), 7.47 (m, 1H), 7.61 (m, 2H), 7.70-7.83 (m, 2H), 8.76 (m, 1H); MS (ESI+) m/z 408 (M+H)⁺; MS (ESI−) m/z 406 (M−H)⁻; Anal. Calcd for C₁₉H₁₉F₆NO₂.0.45 H₂O: C, 54.93; H, 4.83; N, 3.37. Found: C, 54.84; H, 4.64; N, 3.33.

### Example 24

### 3,3-Dimethyl-N-[2-(2,2,2-trifluoro-1-hydroxy-1-trifluoromethyl-ethyl)-naphthalen-1-yl]-butyramide

2-(1-Amino-naphthalen-2-yl)-1,1,1,3,3,3-hexafluoro-propan-2-ol and 3,3-dimethylbutyryl chloride were processed as described in Example 13 to provide the titled compound (18 mg, 9%). ¹H NMR (CDCl₃, 300 MHz) δ1.16 (s, 9H), 2.51 (s, 2H), 7.47 (m, 1H), 7.61 (m, 2H), 7.70-7.84 (m, 2H), 8.71 (m, 1H); MS (ESI+) m/z 408 (M+H)⁺; MS (ESI−) m/z 406 (M−H)⁻; HRMS 408.1385 (408.1398 calcd for C₁₉H₂₀NO₂F₆).

### Example 25

### 2-Methoxy-N-[2-(2.2,2-trifluoro-1-hydroxy-1-trifluoromethyl-ethyl)-naphthalen-1-yl]-acetamide

2-(1-Amino-naphthalen-2-yl)-1,1,1,3,3,3-hexafluoro-propan-2-ol and methoxyacetyl chloride were processed as described in Example 13 to provide the titled compound (150 mg, 61%). mp 120-121° C.; ¹H NMR (CDCl₃, 300 MHz) δ3.60 (s, 3H), 4.15 (s, 2H), 7.60 (m, 2H), 7.72 (d, 1H), 7.90 (m, 2H), 8.80 (m, 1H); MS (ESI+) m/z 382 (M+H)⁺; MS (ESI−) m/z 380 (M−H)⁻; Anal. Calcd for C₁₆H₁₃F₆NO₃: C, 50.40; H, 3.44; N, 3.67. Found: C, 50.69; H, 3.46; N, 3.52.

### Example 26

### Cyclopropanecarboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and cyclopropanecarbonyl chloride were processed as described in Example 13 to provide the titled compound (17 mg, 7%). m.p. 163-165° C.; ¹H NMR (CDCl₃, 300 MHz) rotamers δ0.90-1.25 (m, 4H), 1.90 (m, 1H), 7.45-8.05 (m, 6H); MS (ESI+) m/z 378 (M+H)⁺; MS (ESI−) m/z 376 (M−H)⁻; Anal. Calcd for C₁₇H₁₃F₆NO₂: C, 54.12; H, 3.47; N, 3.71. Found: C, 53.82; H, 3.40; N, 3.43.

### Example 27

### Cyclobutanecarboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and cyclobutanecarbonyl chloride were processed as described in Example 13 to provide the titled compound (33 mg, 13%). m.p. 185-186° C.; ¹H NMR (CDCl₃, 300 MHz) δ2.05 (m, 2H), 2.36 (m, 2H), 2.52 (m, 2H), 3.43 (quintet, 1H), 7.46 (d, 1H), 7.61 (m, 2H), 7.90 (m, 2H), 8.73 (m, 1H); MS (ESI+) m/z 392 (M+H)⁺; MS (ESI−) m/z 390 (M−H)⁻; Anal. Calcd for C₁₈H₁₅F₆NO₂: C, 55.23; H, 3.86; N, 3.58. Found: C, 55.15; H, 3.75; N, 3.41.

### Example 28

### Cyclopentanecarboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and cyclopentanecarbonyl chloride were processed as described in Example 13 to provide the titled compound (13 mg, 5%). ¹H NMR (CDCl₃, 300 MHz) rotamers δ1.55-2.20 (m, 8H), 3.02 (quintet, 1H), 7.46 (d, 1H), 7.60 (m, 2H), 7.88 (m, 2H), 8.72 (m, 1H); MS (ESI+) m/z 406 (M+H)⁺; MS (ESI−) m/z 404 (M−H)⁻; Anal. Calcd for C₁₉H₁₇F₆NO₂: C, 56.30; H, 4.23; N, 3.46. Found: C, 56.57; H, 4.18; N, 3.46.

### Example 29

### 2-Cyclopentyl-N-[2-(2,2,2-trifluoro-1-hydroxy-1-trifluoromethyl-ethyl)-naphthalen-1-yl]-acetamide

2-(1-Amino-naphthalen-2-yl)-1,1,1,3,3,3-hexafluoro-propan-2-ol and cyclopentylacetyl chloride were processed as described in Example 13 to provide the titled compound (81 mg, 30%). m.p. 144-145° C.; ¹H NMR (CDCl₃, 300 MHz) δ1.32 (m, 2H), 1.62 (m, 4H), 1.92 (m, 2H), 2.42 (m, 1H), 2.61 (d, 2H), 7.46 (d1H), 7.60 (m, 2H), 7.88 (m, 2H), 8.72 (m, 1H); MS (ESI+) m/z 420 (M+H)⁺; MS (ESI−) m/z 418 (M−H)⁻; Anal. Calcd for C₂₀H₁₉F₆NO₂+0.05C₇H₈: C, 57.65; H, 4.61; N, 3.30. Found: C, 57.87; H, 4.37; N, 3.08.

### Example 30

### Cyclohexanecarboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and cyclohexanecarbonyl chloride were processed as described in Example 13 to provide the titled compound (42 mg, 20%). ¹H NMR (CDCl₃, 300 MHz) δ1.20-1.92 (m, 6H), 2.08 (m, 4H), 2.40 (m, 1H), 7.18 (s, 1H), 7.38 (d, 1H), 7.54 (m, 2H), 7.74-7.84 (m, 3H); MS (ESI+) m/z 420 (M+H)⁺; MS (ESI−) m/z 418 (M−H)⁻;

### Example 31

### 3-Phenyl-N-[2-(2,2,2-trifluoro-1-hydroxy-1-trifluoromethyl-ethyl -naphthalen-1-yl]-propionamide

2-(1-Amino-naphthalen-2-yl)-1,1,1,3,3,3-hexafluoro-propan-2-ol and hydrocinnamoyl chloride were processed as described in Example 13 to provide the titled compound (43 mg, 15%) as an oil. ¹H NMR (CDCl₃, 300 MHz) δ2.93 (dd, 2H), 3.17 (dd, 2H), 3.44 (s, 1H), 7.23 (m, 1H), 7.29 (m, 4H), 7.48 (m, 1H), 7.60 (m, 2H), 7.77 (m, 1H), 7.83 (m, 1H), 8.72 (m, 1H); MS (ESI+) m/z 442 (M+H)⁺; MS (ESI−) m/z 440 (M−H)⁻; Anal. Calcd for C₂₂H₁₇F₆NO₂+0.25C₇H₈: C, 61.42; H, 4.08; N, 3.06. Found: C, 61.79; H, 3.68; N, 3.34.

### Example 32

### N-[2-(2,2,2-Trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and benzoyl chloride were processed as described in Example 13 to provide the titled compound (142 mg, 53%). m.p. 190-192° C.; ¹H NMR (CDCl₃, 300 MHz) δ5.18 (s, 1H), 7.60 (m, 4H), 7.74 (d, 1H), 7.93 (m, 2H), 7.69 (d, 1H), 8.00 (d, 1H), 8.06 (m, 2H), 8.65 (s, 1H); MS (ESI+) m/z 414 (M+H)⁺; MS (ESI−) m/z 412 (M−H)⁻; Anal. Calcd for C₂₀H₁₃F₆NO₂: C, 58.12; H, 3.17; N, 3.39. Found: C, 58.00; H, 2.94; N, 3.45.

### Example 33

### 2-Methyl-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and o-toluoyl chloride were processed as described in Example 13 to provide the titled compound (28 mg, 10%). ¹H NMR (CDCl₃, 300 MHz) δ2.58 (s, 3H), 7.25-7.42 (m, 3H), 7.58 (m, 2H), 7.69 (d, 1H), 7.84 (m, 2H), 8.03 (m, 1H); MS (ESI+) m/z 428 (M+H)⁺; MS (ESI−) m/z 426 (M−H)⁻; Anal. Calcd for C₂₁H₁₅F₆NO₂: C, 59.02; H, 3.54; N, 3.28. Found: C, 58.91; H, 3.46; N, 3.44.

### Example 34

### 3-Methyl-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and m-toluoyl chloride were processed as described in Example 13 to provide the titled compound (38 mg, 14%). mp 167-168° C.; ¹H NMR (CDCl₃, 300 MHz) δ2.43 (s, 3H), 5.38 (br s, 1H), 7.32 (d, 2H), 7.58 (m, 2H), 7.69 (d, 1H), 7.90 (m, 4H), 8.54 (s, 1H); MS (ESI+) m/z 428 (M+H)⁺; MS (ESI−) m/z 426 (M−H)⁻; Anal. Calcd for C₂₁H₁₅F₆NO₂: C, 59.02; H, 3.54; N, 3.28. Found: C, 58.77; H, 3.44; N, 3.20.

### Example 35

### 4-Methyl-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and p-toluoyl chloride were processed as described in Example 13 to provide the titled compound (45 mg, 16%). ¹H NMR (CDCl₃, 300 MHz) δ2.43 (s, 3H), 5.23 (br s, 1H), 7.41 (d, 2H), 7.58 (m, 2H), 7.69 (d, 1H), 7.80-8.00 (m, 4H), 8.54 (s, 1H); MS (ESI+) m/z 428 (M+H)⁺; MS (ESI−) m/z 426 (M−H)⁻; Anal. Calcd for C₂₁H₁₅F₆NO₂: C, 59.02; H, 3.54; N, 3.28. Found: C, 58.70; H, 3.45; N, 3.46.

### Example 36

### 4-Fluoro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 4-fluorobenzoyl chloride were processed as described in Example 13 to provide the titled compound (61 mg, 22%). mp 135-136° C.; ¹H NMR (CDCl₃, 300 MHz) δ7.20 (m, 2H), 7.52-7.78 (m, 4H), 7.84-8.04 (m, 3H), 8.62 (s, 1H); MS (ESI+) m/z 432 (M+H)⁺; MS (ESI−) m/z 430 (M−H)⁻; Anal. Calcd for C₂₀H₁₂F₇NO₂+0.05 CH₃CN+1.35H₂O: C, 52.75; H, 3.27; N, 3.21. Found: C, 52.51; H, 2.93; N, 3.50.

### Example 37

### N-[2-(2,2,2-Trifluoro-1-hydroxy-1-trifluoromethyl-ethyl)-naphthalen-1-yl]-4-trifluoromethyl-benzamide

2-(1-Amino-naphthalen-2-yl)-1,1,1,3,3,3-hexafluoro-propan-2-ol and 4-trifluoromethylbenzoyl chloride were processed as described in Example 13 to provide the titled compound (78 mg, 25%). mp 191-192° C; ¹H NMR (CDCl₃, 300 MHz) δ4.58 (s, 1H), 7.60 (m, 2H), 7.70 (d, 1H), 7.81 (m, 2H), 7.90 (m, 2H), 8.12 (m, 2H), 8.80 (s, 1); MS (ESI+) m/z 482 (M+H)⁺; MS (ESI−) m/z 480 (M−H)⁻; Anal. Calcd for C₂₁H₁₂F₉NO₂: C, 52.40; H, 2.51; N, 2.91. Found: C, 52.24; H, 2.38; N, 2.78.

### Example 38

### 4-Bromo-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 4-bromobenzoyl chloride were processed as described in Example 13 to provide the titled compound (60 mg, 19%). mp 155-156° C.; ¹H NMR (CDCl₃, 300 MHz) δ4.80 (s, 1H), 7.60 (m, 2H), 7.68 (m, 3H), 7.88 (m, 5H), 8.65 (s, 1H); MS (ESI+) m/z 494 (M+H)⁺; MS (ESI−) m/z 492 (M−H)⁻; Anal. Calcd for C₂₀H₁₂BrF₆NO₂: C, 48.80; H, 2.46; N, 2.85. Found: C, 48.75; H, 2.71; N, 2.76.

### Example 39

### Biphenyl-4-carboxylic acid [2-(2,2,2-trifluoro-1-hydroxy-1-trifluoromethyl-ethyl) naphthalen-1-yl]-amide

2-(1-Amino-naphthalen-2-yl)-1,1,1,3,3,3-hexafluoro-propan-2-ol and 4-phenylbenzoyl chloride were processed as described in Example 13 to provide the titled compound (19 mg, 6%). ¹H NMR (CDCl₃, 300 MHz) δ5.00 (s, 1H), 7.32-8.15 (m, 16H); MS (ESI+) m/z 490 (M+H)⁺; MS (ESI−) m/z 488 (M−H)⁻;

### Example 40

### N-[2-(2,2,2-Trifluoro-1-hydroxy-1-trifluoromethyl-ethyl)-naphthalen-1-yl]-4-trifluoromethoxy-benzamide

2-(1-Amino-naphthalen-2-yl)-1,1,1,3,3,3-hexafluoro-propan-2-ol and 4-trifluoromethoxybenzoyl chloride were processed as described in Example 13 to provide the titled compound (125 mg, 39%). m.p. 179-180° C.; ¹H NMR (CDCl₃, 300 MHz) δ4.93 (s, 1H), 7.32 (d, 2H), 7.58 (m, 2H), 7.65 (d, 1H), 7.84 (m, 3H), 8.02 (m, 2H), 8.65 (s, 1H); MS (ESI+) m/z 498 (M+H)⁺; MS (ESI−) m/z 496 (M−H)⁻; Anal. Calcd for C₂₁H₁₂F₉NO₃: C, 50.72; H, 2.43; N, 2.82. Found: C, 50.45; H, 2.42; N, 2.65.

### Example 41

### Isoxazole-5-carboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and isoxazole-5-carbonyl chloride were processed as described in Example 13 to provide the titled compound (125 mg, 48%). m.p. 162-164° C.; ¹H NMR (CDCl₃, 300 MHz) δ4.64 (s, 1H), 7.09 (s, 1H), 7.62 (m, 2H), 7.70 (d, 1H), 7.95 (m, 3H), 8.44 (s, 1H), 9.44 (s, 1H); MS (ESI+) m/z 405 (M+H)⁺; MS (ESI−) m/z 403 (M−H)⁻; Anal. Calcd for C₁₇H₁₀F₆N₂O₃: C, 50.51; H, 2.49; N, 6.93. Found: C, 50.33; H, 2.57; N, 7.15.

### Example 42

### N-[2-(2,2,2-Trifluoro-1-hydroxy-1-trifluoromethyl-ethyl)-naphthalen-1-yl]-nicotinamide

2-(1-Amino-naphthalen-2-yl)-1,1,1,3,3,3-hexafluoro-propan-2-ol and nicotinoyl chloride were processed as described in Example 13 to provide the titled compound (67 mg, 25%). m.p. 214-215° C.; ¹H NMR (CDCl₃, 300 MHz) δ7.55 (m, 4H), 7.70 (d, 1H), 7.84 (m, 3H), 8.58 (m, 2H), 9.20 (s, 1H), 10.56 (s, 1H); MS (ESI+) m/z 415 (M+H)⁺; MS (ESI−) m/z 413 (M−H)−; Anal. Calcd for C₁₉H₁₂F₆N₂O₂: C, 55.08; H, 2.92; N, 6.76. Found: C, 54.78; H, 2.89; N, 6.71.

### Example 43

### Benzo[b]thiophene-2-carboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and benzo[b]thiophene-2-carbonyl chloride were processed as described in Example 13 to provide the titled compound (127 mg, 42%). m.p. 132-134° C.; ¹H NMR (CDCl₃, 300 MHz) δ5.02 (s, 1H), 7.45 (m, 2H), 7.58 (m, 2H), 7.68 (d, 1H), 7.87 (m, 4H), 7.98 (m, 1H), 8.02 (s, 1H), 8.78 (s, 1H); MS (ESI+) m/z 470 (M+H)⁺; MS (ESI−) m/z 468 (M−H)⁻; Anal. Calcd for C₂₂H₁₃F₆NO₂S+0.35CH₃CN: C, 56.36; H, 2.93; N, 3.91. Found: C, 56.11; H, 2.95; N, 4.07.

### Example 44

### Quinoxaline-2-carboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and 2-quinoxaloyl chloride were processed as described in Example 11, with the exception of reaction time which was increased from 4 h to 22 h. This provided the titled compound (33 mg, 11%). m.p. >240° C.; ¹H NMR (CDCl₃, 300 MHz) δ7.58 (m, 2H), 7.78 (d, 1H), 7.84 (m, 5H), 8.23 (m, 2H), 9.70 (s, 1H); MS (ESI+) m/z 466 (M+H)⁺; MS (ESI−) m/z 464 (M−H)⁻; Calculated m/z for (M+H⁺) C₂₂H₁₄F₆N₃O₂=466.0990; observed m/z=466.0994.

### Example 45

### 3-Fluoro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 3-fluorobenzoyl chloride were processed as described in Example 13 to provide the titled compound (86 mg, 31%). m.p. 190-192° C.; ¹H NMR (CDCl₃, 300 MHz) δ4.78 (s, 1H), 7.37 (m, 2H), 7.60 (m, 3H), 7.78 (m, 2H), 7.92 (d, 3H), 8.70 (s, 1H); MS (ESI+) m/z 432 (M+H)⁺; MS (ESI−) m/z 430 (M−H)⁻; Anal. Calcd for C₂₀H₁₂F₇NO₂: C, 55.69; H, 2.80; N, 3.25. Found: C, 55.69; H, 2.58; N, 3.27.

### Example 46

### 3-Bromo-N-[2-(2,2,2-trifluoro-1-hydroxy-1-trifluoromethyl-ethyl) -naphthalen-1-yl]-benzamide

2-(1-Amino-naphthalen-2-yl)-1,1,1,3,3,3-hexafluoro-propan-2-ol and 3-bromobenzoyl chloride were processed as described in Example 13 to provide the titled compound (76 mg, 24%). m.p. 184-185° C.; 1H NMR (CDCl₃, 300 MHz) δ4.84 (s, 1H), 1H), 7.43 (m, 1H), 7.61 (m, 2H), 7.76 (m, 2H), 7.92 (m, 4H), 8.20 (s, 1H), 8.75 (s, 1H); MS (ESI+) m/z 2.492 (M⁺H)⁺; MS (ESI−) m/z 490 (M−H)⁻; Anal. Calcd for C₂₀H₁₂BrF₆NO₂: C, 48.80; H.

### 2.46; N, 2.85. Found: C, 48.67; H, 2.26; N, 2.80.

### Example 47

### N-[2-(2,2.2-Trifluoro-1-hydroxy-1-trifluoromethyl-ethyl)-naphthalen-1-yl]-3-trifluoromethyl-benzamide

2-(1-Amino-naphthalen-2-yl)-1,1,1,3,3,3-hexafluoro-propan-2-ol and 3-trifluoromethylbenzoyl chloride were processed as described in Example 13 to provide the titled compound (86 mg, 26%). m.p. 187-188° C.; ¹H NMR (CDCl₃, 300 MHz) δ4.81 (s, 1H), 7.61 (m, 2H), 7.70 (m, 2H), 7.92 (m, 4H), 8.20 (m, 1H), 8.30 (s, 1H); MS (ESI+) m/z 482 (M+H)⁺; MS (ESI−) m/z 480 (M−H)⁻; Anal. Calcd for C₂₁H₁₂F₉NO₂: C, 52.40; H, 2.51; N, 2.91. Found: C, 52.43; H. 2.25; N, 2.87.

### Example 48

### 3-Methoxy-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 3-methoxybenzoyl chloride were processed as described in Example 13 to provide the titled compound (21 mg, 9%). ¹H NMR (CDCl₃, 300 MHz) δ3.92 (s, 3H), 7.20 (m, 1H), 7.46 (m, 2H), 7.62 (m, 3H), 7.78 (m, 1H), 7.93 (m, 2H), 7.98 (m, 1H), 8.62 (s, 1H); MS (ESI+) m/z 444 (M+H)⁺; MS (ESI−) m/z 442 (M−H)⁻; Anal. Calcd for C₂₁H₁₅F₆NO₃+0.1TFA: C, 52.40; H, 2.51; N, 2.91. Found: C, 52.19; H, 2.20; N, 3.08.

### Example 49

### 3-Nitro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 3-nitrobenzoyl chloride were processed as described in Example 13 to provide the titled compound (38 mg, 18%). m.p. 211-212° C.; ¹H NMR (CDCl₃, 300 MHz) δ4.81 (s, 1H), 7.61 (m, 2H), 7.70 (m, 2H), 7.92 (m, 4H), 8.20 (m, 1H), 8.30 (s, 1H); MS (ESI+) m/z 459 (M+H)⁺; MS (ESI−) m/z 457 (M−H)−; Anal. Calcd for C₂₁H₁₂F₉NO₂: C, 52.40; H, 2.51; N, 2.91. Found: C, 52.43; H. 2.25; N, 2.87.

### Example 50

### 3-Cyano-N-[2-(2,2,2-trifluoro-1-hydroxy-1-tryfluoromethyl-ethyl -naphthalen-1-yl]-benzamide

2-(1-Amino-naphthalen-2-yl)-1,1,1,3,3,3-hexafluoro-propan-2-ol and 3-cyanobenzoyl chloride were processed as described in Example 13 to provide the titled compound (56 mg, 20%). mp 186-187° C. ¹H NMR (CDCl₃, 300 MHz) δ3.46 (s, 1H), 7.61 (m, 4H), 7.88 (m, 4H), 8.24 (m, 2H), 9.13 (br s, 1H); MS (ESI) m/z 456 (M+NH₄)⁺; MS (ESI) m/z 437 (M−H)⁻; Anal. Calcd for C₂₁H₁₂F₆N₂O₂: C, 57.54; H, 2.76; N, 6.39. Found: C, 57.15; H, 2.84; N, 6.29.

### Example 51

### 4-Iodo-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 4-iodobenzoyl chloride were processed as described in Example 13 to provide the titled compound (44 mg, 13%). mp 189-190° C. ¹H NMR (CDCl₃, 300 MHz) δ2.00 (s, 1H), 7.56 (m, 2H), 7.68 (m, 2H), 7.86 (m, 5H), 8.81 (br s, 1H); MS (ESI) m/z 540 (M+H)⁺; MS (ESI) m/z 538 (M−H)⁻; Anal. Calcd for C₂₀H₁₂F₆INO₂: C, 44.55; H, 2.24; N, 2.60. Found: C, 44.16; H, 2.44; N, 2.51.

### Example 52

### Furan-2-carboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and furan-2-carbonyl chloride were processed as described in Example 11. The resulting bis substituted product was treated with K₂CO₃ in methanol for 24 hours. The resulting mixture was filtered and solvent was removed. The resulting residue was then purified by reverse-phase chromatography to provide the titled compound (23 mg, 43%). mp 228-229° C. ¹H NMR (CDCl₃ & CD₃OD, 300 MHz) δ6.61 (dd, 1H), 7.27 (dd, 1H), 7.57 (m, 3H), 7.71 (2×br s, 1H), 7.89 (m, 3H); MS (ESI) m/z 404 (M+H)⁺; MS (ESI) m/z 402 (M−H)⁻; Anal. Calcd for C₁₈H₁₁F₆NO₃: C, 53.61; H, 2.75; N, 3.47. Found: C, 53.54; H, 2.82; N, 3.80.

### Example 53

### Thiophene-2-carboxylic acid [2-(2,2,2-trifluoro-1-hydroxy-1-trifluoromethyl-ethyl)-naphthalen-1-yl]-amide

2-(1-Amino-naphthalen-2-yl)-1,1,1,3,3,3-hexafluoro-propan-2-ol and thiophene-2-carbonyl chloride were processed as described in Example 13 to provide the titled compound (11 mg, 4%). mp 191-192° C. ¹H NMR (CDCl₃, 300 MHz) δ5.02 (s, 1H), 7.21 (m, 1H), 7.78 (m, 8H), 8.48 (m, 1H); MS (ESI) m/z 420 (M+H)⁺; MS (ESI) m/z 418 (M−H)⁻; Anal. Calcd for C₁₈H₁₁F₆NO₂S: C, 51.56; H, 2.64; N, 3.34. Found: C, 51.36; H, 2.57; N, 3.44.

### Example 54

### 2,3-Dichloro-N-[2-(2,2,2-trifluoro-1-hydroxy-1-trifluoromethyl-ethyl -naphthalen-1-yl]-benzamide

2-(1-Amino-naphthalen-2-yl)-1,1,1,3,3,3-hexafluoro-propan-2-ol and 2,3-dichlorobenzoyl chloride were processed as described in Example 13 to provide the titled compound (42 mg, 14%). mp 199-202° C. ¹H NMR (CDCl₃, 300 MHz) δ4.89 (s, 1H), 7.33 (t, 1H), 7.65 (m, 5H), 7.92 (m, 2H), 8.21 (m, 1H), 8.51 (s, 1H); MS (ESI) m/z 482 (M+H)⁺; MS (ESI) m/z 480 (M−H)⁻; Anal. Calcd for C₂₀H₁₁Cl₂F₆NO₂: C, 49.82; H, 2.30; N, 2.91. Found: C, 49.51; H, 2.45; N, 2.85.

### Example 55

### 3,4-Dichloro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 3,4-dichlorobenzoyl chloride were processed as described in Example 13 to provide the titled compound (42 mg, 13%). mp 174-175° C. ¹H NMR (CDCl₃, 300 MHz) δ5.02 (s, 1H), 7.57 (m, 4H), 7.67 (2×br s, 1H), 7.76 (m, 1H) ,7.88 (m, 2H), 8.05 (m, 1H), 8.84 (s, 1H); MS (ESI) m/z 482 (M+H)⁺; MS (ESI) m/z 480 (M−H)⁻; Anal. Calcd for C₂₀H₁₁Cl₂F₆NO₂: C, 49.82; H, 2.30; N, 2.91. Found: C, 49.67; H, 2.26; N, 2.83.

### Example 56

### 3,5-Dichloro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 3,5-dichlorobenzoyl chloride were processed as described in Example 13 to provide the titled compound (46 mg, 14%). mp 205-207° C. ¹H NMR (CDCl₃, 300 MHz) δ4.70 (s, 1H), 7.60 (m, 3H), 7.70 (2×br s, 1H), 7.88 (m, 5H), 8.81 (s, 1H); MS (ESI) m/z 482 (M+H)⁺; MS (ESI) m/z 480 (M−H)⁻; Anal. Calcd for C₂₀H₁₁Cl₂F₆NO₂: C, 49.82; H, 2.30; N, 2.91. Found: C, 49.94; H, 2.36; N, 3.16.

### Example 57

### 4-Chloro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 4-chlorobenzoyl chloride were processed as described in Example 13 to provide the titled compound (55 mg, 19%). mp 175-176° C. ¹H NMR (CDCl₃, 300 MHz) δ5.37 (s, 1H), 7.41 (m, 2H), 7.54 (m, 2H), 7.65 (2×br s, 1H), 7.85 (m, 5H), 8.83 (s, 1H); MS (ESI) m/z 448 (M+H)⁺; MS (ESI) m/z 446 (M−H)⁻; Anal. Calcd for C₂₀H₁₂ClF₆NO₂: C, 53.65; H, 2.70; N, 3.13. Found: C, 53.52; H, 2.56; N, 3.22.

### Example 58

### 3-Chloro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 3-chlorobenzoyl chloride were processed as described in Example 13 to provide the titled compound (44 mg, 15%). mp 179-181° C. ¹H NMR (CDCl₃, 300 MHz) δ3.93 (s, 0.4H), 4.90 (s, 0.6H), 7.55 (m, 4H), 7.70 (2×br s, 0.6H), 7.93 (m, 4.6H), 8.12 (2×br s, 0.4H), 8.19 (s, 0.4H), 8.72 (s, 0.6H), 9.06 (d, 0.4H); MS (ESI) m/z 448 (M+H)⁺; MS (ESI) m/z 446 (M−H)⁻; Anal. Calcd for C₂₀H₁₂ClF₆NO₂: C, 53.65; H, 2.70; N, 3.13. Found: C, 53.42; H, 2.87; N, 3.36.

### Example 59

### 2-Chloro-N-[2-(2,2,2-trifluoro-1-hydroxy-1-trifluoromethyl-ethyl)-naphthalen-1-yl]-benzamide

2-(1-Amino-naphthalen-2-yl)-1,1,1,3,3,3-hexafluoro-propan-2-ol and 2-chlorobenzoyl chloride were processed as described in Example 13 to provide the titled compound (48 mg, 16%). mp 209-211° C. ¹H NMR (CDCl₃, 300 MHz) δ4.87 (s, 1H), 7.49 (m, 3H), 7.63 (m, 2H), 7.74 (2×br s, 1H), 7.92 (m, 3H), 8.20 (m, 1H), 8.63 (s, 1H); MS (ESI) m/z 448 (M+H)⁺; MS (ESI) m/z 446 (M−H)⁻; Anal. Calcd for C₂₀H₁₂ClF₆NO₂.0.15 H₂O: C, 53.33; H, 2.75; N, 3.11. Found: C, 53.01; H, 2.66; N, 3.43.

### Example 60

### N-[4-Bromo-2-(2,2,2-trifluoro-1-hydroxy-1-trifluoromethyl-ethyl)-naphthalen-1-yl]-acetamide

A-151892 (100 mg, 0.28 mmol) and sodium acetate (30 mg, 0.36 mmol) were dissolved in 1:1 acetic acid-tetrahydrofuran (1 mL). Bromine (58 mg, 20 μL, 0.36 mmol) was added and the suspension stirred at room temperature for 4h. A second equivalent was added and stirring continued for a further 1 hour. Removal of solvents at pump was followed by partitioning between ethyl acetate and water. The organic layer was washed with brine, dried (NgSO₄) and separated by reverse-phase chromatography to provide the titled compound (45 mg, 37%). ¹H NMR (CDCl₃, 300 MHz) rotamers δ2.36, 2.39 (s, 3H), 7.60-7.80 (m, 3H), 8.22 (d, 1H), 8.74 (d, 1H); MS (ESI+) m/z 432 (M+H)⁺; MS (ESI−) m/z 430 (M−H)⁻; Anal. Calcd for C₁₅H₁₀BrF₆NO₂: C, 41.88; H, 2.34; N, 3.26. Found: C, 41.86; H, 2.23; N, 3.06.

### Example 61

### 1-(4-Chloro-phenyl)-3- [2-(2,2,2-trifluoro-1-hydroxy-1-trifluoromethyl-ethyl)-naphthalen-1-yl]-urea

To a solution of 2-(1-amino-naphthalen-2-yl)-1,1,1,3,3,3-hexafluoro-propan-2-ol (149 mg, 0.48 mmol) in diethyl ether (5 mL), a solution of 4-chlorophenyl isocyanate (87 mg, 0.57 mmol) in diethyl ether (5 mL) was added dropwise over 2 minutes. After the addition was complete the reaction solution was stirred for 2 hours at room temperature. The solvent was removed at the pump and the residue recrystallized from hot ether to give the titled compound (60 mg, 27%). mp 210-212° C. ¹H NMR (CDCl₃, 300 MHz) δ1.25 (s, 1H), 6.40 (s, 1H) 7.04 (s, 1H), 7.13 (m, 4H), 7.65 (m, 2H), 7.80 (d, 1H), 7.95 (m, 2H), 8.11 (m, 1H); MS (ESI+) m/z 463 (M+H)⁺; MS (ESI−) m/z 460 (M−H)⁻; Anal. Calcd for C₂₀H₁₃ClF₆N₂O₂: C, 51.91; H, 2.83; N, 6.05. Found: C, 52.21; H, 2.94; N, 6.07.

### Example 62

### [2-(2,2,2-Trifluoro-1-hydroxy-1-trifluoromethyl-ethyl)-naphthalen-1-yl]-carbamic acid isopropyl ester

To a solution of 2-(1-amino-naphthalen-2-yl)-1,1,1,3,3,3-hexafluoro-propan-2-ol (150 mg, 0.49 mmol) in pyridine (3 mL) isopropylchloroformate (1 M in toluene, 0.53 mL, 0.53 mmol) was added. The mixture was stirred at room temperature for 24 hours. The pyridine was removed at the pump and the resulting residue was purified by reverse-phase chromatography to provide the titled compound (16 mg, 8%). mp 139-141° C. ¹H NMR (CDCl₃, 300 MHz) rotamers: δ1.23, 1.35 (d, 6H), 5.06 (m, 11H), 6.91, 7.00 (s, 1H), 7.59 (m, 2H), 7.71 (d, 1H), 7.88 (m, 2H), 7.97 (m, 1H); MS (ESI+) m/z 396 (M+H)⁺; MS (ESI−) m/z 394 (M−H)⁻.

### Biological Data

### Determination of Potassium Channel Opening Activity Membrane Hyperpolarization Assays

Compounds were evaluated for potassium channel opening activity using primary cultured guinea-pig urinary bladder (GPB) cells. In the preparation of urinary bladder smooth muscle cells, urinary bladders were removed from male guinea-pigs (Hartley, Charles River, Wilmington, Mass.) weighing 300-400 g and placed in ice-cold Ca²⁺-free Krebs solution (Composition, mM: KCl, 2.7; KH₂PO₄, 1.5; NaCl, 75; Na₂HPO₄, 9.6; Na₂HPO₄.7H₂O, 8; MgSO₄, 2; glucose, 5; HEPES, 10; pH 7.4). Cells were isolated by enzymatic dissociation as previously described with minor modifications (Klockner, U. and Isenberg, G., Pflugers Arch. (1985), 405, 329-339), hereby incorporated by reference. The bladder was cut into small sections and incubated in 5 mL of the Kreb's solution containing 1 mg/mL collagenase (Sigma, St. Louis, Mo.) and 0.2 mg/mL pronase (Calbiochem, La Jolla, Calif.) with continuous stirring in a cell incubator for 30 minutes. The mixture was then centrifuged at 1300×g for 5 minutes, and the pellet resuspended in Dulbecco's PBS (GIBCO, Gaithersburg, Md.) and recentrifuged to remove residual enzyme. The cell pellet was resuspended in 5 mL growth media (composition: Dulbecco's modified Eagle's medium supplemented with 10% fetal bovine serum, 100 units/mL penicillin, 100 units/mL streptomycin and 0.25 mg/mL amphotericin B) and further dissociated by pipetting the suspension through a flame-polished Pasteur pipette and passing it through a polypropylene mesh membrane (Spectrum, Houston, Tex.). The cell density was adjusted to 100,000 cells/mL by resuspension in growth media. Cells were plated in clear-bottomed black 96-well plates (Packard) for membrane potential studies at a density of 20,000 cells/well and maintained in a cell incubator with 90% air:10% CO₂ until confluent. Cells were confirmed to be of smooth muscle type by cytoskeletal staining using a monoclonal mouse anti human-a-smooth muscle actin (Biomeda, Foster City, Calif.).

Functional activity at potassium channels was measured by evaluating changes in membrane potential using the bis-oxonol dye DiBAC(4)₃ (Molecular Probes) in a 96-well cell-based kinetic assay system, Fluorescent Imaging Plate Reader (FLIPR) (K. S. Schroeder et al., J. Biomed. Screen., v. 1 pp. 75-81 (1996)), hereby incorporated by reference. DiBAC(4)₃ is an anionic potentiometric probe which partitions between cells and extracellular solution in a membrane potential-dependent manner. With increasing membrane potential (for example, K⁺ depolarization there is an increase in fluorescence observed due to dye interaction with intracellular lipids and proteins. Conversely, decreasing membrane potential (hyperpolarization by potassium channel openers) evokes a decrease in fluorescence.

Confluent guinea-pig urinary bladder cells cultured in black clear-bottomed 96-well plates were rinsed twice with 200 mL assay buffer (composition, mM: HEPES, 20; NaCl, 120; KCl, 2; CaCl₂, 2; MgCl₂, 1; glucose, 5; pH 7.4 at 25° C.) containing 5 μM DiBAC(4)₃ and incubated with 180 mL of the buffer in a cell incubator for 30 minutes at 37° C. to ensure dye distribution across the membrane. After recording the baseline fluorescence for 5 minutes, the reference or test compounds, prepared at 10 times the concentration in the assay buffer, were added directly to the wells. Changes in fluorescence were monitored for an additional 25 minutes. Hyperpolarization responses were corrected for any background noise and were normalized to the response observed with 10 μM of the reference compound P1075 (assigned as 100%), a potent opener of smooth muscle K_{ATP} channels (Quast et al., Mol. Pharmacol., v. 43 pp. 474-481 (1993)), hereby incorporated by reference.

Routinely, five concentrations of P1075 or test compounds (log or half-log dilutions) were evaluated and the maximal steady-state hyperpolarization values (expressed as % relative to P1075) plotted as a function of concentration. The EC₅₀ (concentration that elicites 50% of the maximal response for the test sample) values were calculated by non-linear regression analysis using a four parameter sigmoidal equation. The maximal response of each compound (expressed as % relative to P1075) is reported. Stock solutions of compounds were prepared in 100% DMSO and further dilutions were carried out in the assay buffer and added to a 96-well plate.

The compounds of the present invention exhibit a 50% maximal response of membrane hyperpolarization in Guinea Pig Bladder cells (as compared to P1075) at doses >1000 nM. In a prefered range, compounds of the present invention exhibit a 50% maximal response of membrane hyperpolarization of Guinea Pig Bladder cells (as compared to P1075) at doses between 100 nM and 1000 nM. In a most preferred range, compounds of the present invention exhibit a 50% maximal response of membrane hyperpolarization of Guinea Pig Bladder cells (as compared to P1075) at doses less than or equal to 100 nM.

### Membrane Potential Studies Using FLIPR

Changes in membrane potential responses were assessed using the bis-oxonol dye DiBAC₄ (Molecular Probes, Oreg.) in the Fluorometric Imaging Plate Reader (FLIPR, Sunnyvale, Calif.) as detailed previously (Gopalakrishnan et al, J. Pharmacol. Exp. Ther., 1999, 289(1), 551-558). Confluent stably transfected Kir 6.2/SUR2B exon 17+ or SUR2B exon 17− cells (Gopalakrishnan et al, Br. J. Pharmacol., 2000, 129(7), 1323-1332) were plated at a density of 40,000 cells/ well in black, clear-bottomed 96-well plates and assays were carried out in buffer (20 mM HEPES, 120 mM NaCl, 2 mM KCl, 2 mM CaCl₂, 1 mM MgCl₂, 5 μM Glucose, pH 7.4) containing 5 μm DiBAC₄ at 37° C. Changes in fluorescence were monitored at excitation and emission wavelengths of 488 and 520 nm respectively for 25 min. Data was normalized to the response evoked by 10 mm P1075.

The compounds of the present invention exhibit a 50% maximal response of JiS 15 membrane hyperpolarization in transfected Kir 6.2/SUR2B exon 17− cells at doses >1000 nM. In a prefered range, compounds of the present invention exhibit a 50% maximal response of membrane hyperpolarization of transfected Kir 6.2/SUR2B exon 17− cells at doses between 100 nM and 1000 nM. In a most preferred range, compounds of the present invention exhibit a 50% maximal response of membrane hyperpolarization transfected Kir 6.2/SUR2B exon 17-cells at doses less than or equal to 100 nM.

### In Vitro Functional Models

Compounds were evaluated for functional potassium channel opening activity using tissue strips obtained from Landrace pig bladders. Landrace pig bladders were obtained from female Landrace pigs of 9-30 kg. Landrace pigs were euthanized with an intraperitoneal injection of pentobarbital solution, Somlethal®, J. A. Webster Inc., Sterling Mass. The entire bladder was removed and immediately placed into Krebs Ringer bicarbonate solution (composition, mM: NaCl, 120; NaHCO₃, 20; dextrose, 11; KCl, 4.7; CaCl₂, 2.5; MgSO₄, 1.5; KH₂PO₄, 1.2; K₂EDTA, 0.01, equilibrated with 5% CO₂/95% O₂ pH 7.4 at 37° C.). Propranolol (0.004 mM) was included in all of the assays to block β-adrenoceptors. The trigonal and dome portions were discarded. Strips 3-5 mm wide and 20 mm long were prepared from the remaining tissue cut in a circular fashion. The mucosal layer was removed. One end was fixed to a stationary glass rod and the other to a Grass FT03 transducer at a basal preload of 1.0 gram. Two parallel platinum electrodes were included in the stationary glass rod to provide field stimulation of 0.05 Hz, 0.5 milli-seconds at 20 volts. This low frequency stimulation produced a stable twitch response of 100-500 centigrams. Tissues were allowed to equilibrate for at least 60 minutes and primed with 80 mM KCl. A control concentration response curve (cumulative) was generated for each tissue using the potassium channel opener P1075 as the control agonist. P1075 completely eliminated the stimulated twitch in a dose dependent fashion over a concentration range of 10⁻⁹ to 10⁻⁵ M dissolved in DMSO using ½ log increments. After a 60 minute rinsing period, a concentration response curve (cumulative) was generated for the test agonist in the same fashion as that used for the control agonist P1075. The maximal efficacy of each compound (expressed as % relative to P1075) is reported. The amount of agent necessary to cause 50% of the agent's maximal response (ED₅₀) was calculated using “ALLFIT” (DeLean et al., Am. J. Physiol., 235, E97 (1980)), hereby incorporated by reference. Agonist potencies were also expressed as an index relative to P1075. The index was calculated by dividing the ED₅₀ for P1075 by the ED₅₀ for the test agonist in a given tissue. Each tissue was used for only one test agonist, and the indices obtained from each tissue were averaged to provide an average index of potency.

The compounds of the present invention exhibit a 50% maximal response of Functional Potassium Channel Opening Activity in Isolated Bladder Strips at doses >1000 nM. In a prefered range, compounds of the present invention exhibit a 50% Functional Potassium Channel Opening Activity in Isolated Bladder Strips at doses between 100 nM and 1000 nM. In a most preferred range, compounds of the present invention exhibit a 50% maximal response Functional Potassium Channel Opening Activity in Isolated Bladder Strips at doses less than or equal to 100 nM.

As demonstrated by the data, the compounds of the present invention stimulate contractions of the bladder by opening potassium channels and therefore have utility in the treatment of diseases prevented by or ameliorated with potassium channel openers.

The present invention provides pharmaceutical compositions which comprise compounds of formula (I) prepared and formulated together with one or more non-toxic pharmaceutically acceptable carriers. The pharmaceutical compositions can be formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrastemal, subcutaneous, intraarticular injection and infusion.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin); f) absorption accelerators such as quaternary ammonium compounds; g) wetting agents such as cetyl alcohol and glycerol monostearate;) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

When used in the above or other treatments, a therapeutically effective amount of one of the compounds of the present invention can be employed in pure form or, where such forms exist, in pharmaceutically acceptable salt, ester or prodrug form. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The compounds of the present invention can be used in the form of pharmaceutically acceptable salts derived from inorganic or organic acids. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts are well-known in the art. For example, S. M. Berge et al. describe pharmaceutically acceptable salts in detail in (J. Pharmaceutical Sciences, 1977, 66: 1 et seq). The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quatemized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

The term “pharmaceutically acceptable prodrug” or “prodrug,”as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I), for example, by hydrolysis in blood.

Typical examples of “pharmaceutically acceptable prodrug” or “prodrug” as used herein, can be made as either ester or other groups know to those skilled in the art that may be hydrolyzed under physiological conditions thus delivering a compound of formula (I). Examples of prodrug ester groups include pivoyloxymethyl, acetoxymethyl, phthalidyl, indanyl and methoxymethyl, as well as other such groups known in the art. Other examples of prodrug ester groups can be found in the book (“Pro-drugs as Novel Delivery Systems,” by Higuchi and Stella) cited above. The present invention contemplates pharmaceutically acceptable prodrug esters formed by the attachment of a prodrug ester group as defined herein to X of a compound of formula (I), wherein X is OH.

The present invention contemplates pharmaceutically active metabolites formed by in vivo biotransformation of compounds of formula (I). The term pharmaceutically active metabolite, as used herein, refers to a compound formed by the in vivo biotransformation of compounds of formula (I). The present invention contemplates compounds of formula (I) and metabolites thereof. A thorough discussion of biotransformation is provided in Goodman and Gilman's, The Pharmacological Basis of Therapeutics, seventh edition, hereby incorporated by reference.

The compounds of the invention, including but not limited to those specified in the examples, possess potassium channel opening activity in mammals (especially humans). As potassium channel openers, the compounds of the present invention are useful for the treatment and prevention of diseases such as asthma, epilepsy, Raynaud's syndrome, impotence, migraine, pain, eating disorders, urinary incontinence, functional bowel disorders, neurodegeneration and stroke.

Actual dosage levels of active ingredients in the pharmaceutical compositions of this invention can be varied so as to obtain an amount of the active compound(s) which achieves the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

The total daily dose of the compounds of this invention administered to a human or lower animal may range from about 0.003 to about 10 mg/kg/day. For purposes of oral administration, more preferable doses can be in the range of from about 0.01 to about 5 mg/kg/day. If desired, the effective daily dose can be divided into multiple doses for purposes of administration; consequently, single dose compositions may contain such amounts or submultiples thereof to make up the daily dose.

Potassium channel openers (KCOs) have been shown to act as smooth muscle relaxants, to hyperpolarize bladder cells and consequently relax bladder smooth muscle cells. Because bladder overactivity and urinary incontinence can result from the spontaneous, uncontrolled contractions of the smooth muscle of the bladder, the ability of potassium channel openers to hyperpolarize bladder cells and relax bladder smooth muscle may provide a method to ameliorate or prevent bladder overactivity. Potassium channel openers have been shown to be useful in the treatment of bladder overactivity, pollakiuria, bladder instability, nocturia, bladder hyperreflexia, urinary incontinence, and enuresis as reported by Andersson, et al., Urology 1997, 50 (Suppl 6A), 74-84; Lawson, et al., Pharmacol. Ther. 1996, 70, 39-63; Nurse, et al., Br. J. Urol., 1991, 68, 27-31; Howe, et al., J. Pharmacol. Exp. Ther., 1995, 274, 884-890; Gopalakrishnan, et al., Drug Development Research, 1993, 28, 95-127. Therefore, the compounds of the present invention, including but not limited to those specified in the examples can be used in the treatment of bladder overactivity, pollakiuria, bladder instability, nocturia, bladder hyperreflexia, urinary incontinence, and enuresis.

It has been shown in the past that neuronal hyperpolarization can produce analgesic effects. In fact, the opening of potassium channels and the resultanting hyperpolarization in the membrane of target neurons is a key mechanism in the effect of opioids. The peripheral antinociceptive effect of morphine results from activation of ATP-sensitive potassium channels, which causes hyperpolarization of peripheral terminals of primary afferents, leading to a decrease in action potential generation has been reported by Rodrigues, Br., et al., J Pharmacol 2000, 129(1), 110-4. Opening of K_{ATP} channels by potassium channel openers plays an important role in the antinociception mediated by alpha-2 adrenoceptors and mu opioid receptors. KCO's can also potentiate the analgesic action of both morphine and dexmedetomidine via an activation of K_{ATP} channels at the spinal cord level as reported by Vergoni, et al., Life Sci. 1992, 50(16), PL135-8; Asano, et al., Anesth. Analg. 2000, 90(5), 1146-51. Potassium channel openers therefore are useful as analgesics in the treatment of various pain states including but not limited to migraine and dyspareunia as reported by by Lawson, et al., Pharmacol. Ther. 1996, 70, 39-63; Gopalakrishnan, et al., Drug Development Research, 1993, 28, 95-127; Gehlert, et al., Prog. Neuro-Psychopharmacol. & Biol. Psychiat., 1994, 18, 1093-1102. Therefore, the compounds of the present invention, including but not limited to those specified in the examples can be useful as analgesics in the treatment of various pain states including but not limited to migraine and dyspareunia.

The irritative symptoms of BPH (urgency, frequency, nocturia and urge incontinence) have been shown to be correlated to bladder instability as reported by Pandita, et al., The J. of Urology 1999, 162, 943; and treated using potassium channel openers as reported by Andersson; et al., Prostate 1997, 30, 202-215. Therefore, compounds of the present invention, including but not limited to those specified in the examples, can be used to hyperpolarize bladder cells and relax bladder smooth muscle providing a method to ameliorate or prevent the symptoms associated with BPH.

The excitability of corpus cavernosum smooth muscle cells is important in the male erectile process. The relaxation of corporal smooth muscle cells allows arterial blood to build up under pressure in the erectile tissue of the penis leading to erection Andersson, et al., Pharmacological Reviews 1993, 45, 253. Potassium channels play a significant role in modulating human corporal smooth muscle tone, and thus, erectile capacity. Potassium channel openers are smooth muscle relaxants and have been shown to relax corpus cavernosal smooth muscle and induce erections as reported by Andersson, et al., Pharmacological Reviews 1993, 45, 253; Lawson, et al., Pharmacol. Ther., 1996, 70, 39-63, Vick, et al., J. Urol. 2000, 163: 202. Therefore, the compounds of the present invention, including but not limited to those specified in the examples, can be used in the treatment of male sexual dysfunctions such as male erectile dysfunction, impotence and premature ejaculation.

Sexual arousal and excitement are linked to the blood flow to the genital area and lubricate the vagina as a result of plasma transudation. Topical application of KCOs like minoxidil and nicorandil have been shown to increase clitoral blood flow as reported by Kim, J. J., Yu, J. W., Lee, J. G., Moon, D. G., “Effects of topical K-ATP channel opener solution on clitoral blood flow”, J. Urol. 2000, 163 (4), 240. KCO's can be effective for the treatment of female sexual dysfunction including clitoral erectile insufficiency, vaginismus and vaginal engorgement as mentioned in Goldstein, I. and Berman, J. R., “Vasculogenic female sexual dysfunction: vaginal engorgement and clitoral erectile insufficiency syndromes”, Int. J. Impotence Res. 1998, 10, S84—S90, as they increase blood flow to female sexual organs. Therefore, the compounds of the present invention, including but not limited to those specified in the examples, can be used in the treatment of femal sexual dysfunction as described herein.

Potassium channel openers may have utility as tocolytic agents to inhibit uterine contractions to delay or prevent premature parturition in individuals or to slow or arrest delivery for brief periods to undertake other therapeutic measures as described in Sanborn, et al., Semin. Perinatol., 1995, 19, 31-40; Morrison, et al., Am. J. Obstet. Gynecol., 1993, 169(5), 1277-85. Potassium channel openers also inhibit contractile responses of human uterus and intrauterine vasculature. This combined effect would suggest the potential use of KCO's for dysmenhorrea as mentioned in Kostrzewska, et al., Acta Obstet. Gynecol. Scand., 1996, 75(10), 886-91. Therefore, since the compounds of the present invention, including but not limited to those specified in the examples relax uterine smooth muscle and intrauterine vasculature they can have utility in the treatment of premature labor and dysmenorrhoea as suggested by Lawson, et al., Pharmacol. Ther., 1996, 70, 39-63.

Potassium channel openers have also been shown to relax gastrointestinal smooth tissues and useful in the treatment of functional bowel disorders such as irritable bowel syndrome Lawson, et al., Pharmacol. Ther., 1996, 70, 39-63. Therefore the compounds of the present invention, including but not limites to those specified in the examples are useful in the treatment of functional bowel disorders such as irritable bowel syndrome.

Potassium channel openers relax airway smooth muscle and induce bronchodilation and are useful in the treatment of asthma and airways hyperreactivity as mentioned by Lawson, et al., Pharmacol. Ther., 1996, 70, 39-63; Buchheit, et al., Pulmonary Pharmacology & Therapeutics 1999, 12, 103; Gopalakrishnan, et al., Drug Development Research, 1993, 28, 95-127. Therefore, the compounds of the present invention, including but not limited to those specified in the examples are useful in the treatment of asthma and airways hyperreactivity.

Epilepsy results from the propagation of nonphysiologic electrical impulses. Potassium channel openers hyperpolarize neuronal cells and lead to a decrease in cellular excitability and have demonstrated antiepileptic effects as demonstrated by Lawson, et al., Pharmacol. Ther., 1996, 70, 39-63; Gopalakrishnan, et al., Drug Development Research, 1993, 28, 95-127; Gehlert, et al., Prog. Neuro-Psychopharmacol. & Biol. Psychiat., 1994, 18, 1093-1102. Therefore, the compounds of the present invention, including but not limited to those specified in the examples can be useful in the treatment of epilepsy.

Neuronal cell depolarization can lead to excitotoxicity and neuronal cell death. When this occurs as a result of acute ischemic conditions, the result is often stroke. Long-term neurodegeneration can bring about conditions such as Alzheimer's and Parkinson's diseases. Potassium channel openers can hyperpolarize neuronal cells and lead to a decrease in cellular excitability. Activation of potassium channels has been shown to enhance neuronal survival. Potasium channel openers have been shown to have utility as neuroprotectants in the treatment of neurodegenerative conditions and diseases such as cerebral ischemia, stroke, Alzheimer's disease and Parkinson's disease as mentioned in Lawson, et al., Pharmacol. Ther., 1996, 70, 39-63; Gopalakrishnan, et al., Drug Development Research, 1993, 28, 95-127; Gehlert, et al., Prog. Neuro-Psychopharmacol & Biol. Psychiat., 1994, 18, 1093-1102; Freedman, et al., The Neuroscientist 1996, 2, 145. Therefore, the compounds of the present invention, including but not limited to those specified in the examples will have utility as neuroprotectants in the treatment of neurodegenerative conditions and diseases such as cerebral ischemia, stroke, Alzheimer's disease and Parkinson's disease.

Potassium channel openers also have utility in the treatment of diseases or conditions associated with decreased skeletal muscle blood flow. These conditions are most often associated with diseases associated with decreased skeletal muscle blood flow such as Raynaud's syndrome and intermittent claudication as described in Lawson, et al., Pharmacol. Ther., 1996, 70, 39-63; Gopalakrishnan, et al., Drug Development Research, 1993, 28, 95-127; Dompeling, et al., Vasa. Supplementum, 1992, 3434; and WO9932495. Therefore, the compounds of the present invention, including but not limited to those specified in the examples may be used in the treatment of diseases associated with decreased skeletal muscle blood flow such as Raynaud's syndrome and intermittent claudication

Potassium channel openers have been shown to be useful in the treatment of eating disorders such as obesity Spanswick, et al., Nature, 1997, 390, 521-25; Freedman, et al., The Neuroscientist, 1996, 2, 145. Therefore the compounds of the present invention, including but not limited to those specified in the examples can be useful in the treatment of eating disorders such as obesity.

Potassium channel openers have been shown to promote hair growth as reported by Lawson, et al., Pharmacol. Ther., 1996, 70, 39-63; Gopalakrishnan, et al., Drug Development Research, 1993, 28, 95-127. Therefore, the compounds of the present invention, including but not limited to those specified in the examples can have utility in the treatment of hair loss and baldness also known as alopecia

Potassium channel openers possess cardioprotective effects against myocardial injury during ischemia and reperfusion as mentioned in Garlid, et al., Circ. Res., 1997, 81(6), 1072-82, and have demonstrated an ability to be useful in the treatment of heart diseases Lawson, et al., Pharmacol. Ther., 1996, 70, 39-63; Grover, et al., J. Mol. Cell Cardiol,. 2000, 32, 677. Therefore, the compounds of the present invention, including but not limited to those specified in the examples can be useful in the treatment of heart diseases.

Potassium channel openers, by hyperpolarization of smooth muscle membranes, can exert vasodilation of the collateral circulation of the coronary vasculature leading to increase blood flow to ischemic areas and are thus useful for the treatment of useful for the coronary artery disease as described in Lawson, et al., Pharmacol. Ther., 1996, 70, 39-63; Gopalakrishnan, et al., Drug Development Research, 1993, 28, 95-127. Therefore, the compounds of the present invention, including but not limited to those specified in the examples can be useful for the coronary artery disease.

What is claimed is:

1. A compound of formula (1): 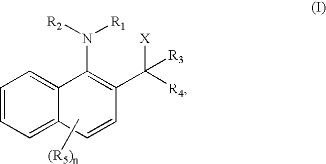
or a pharmaceutically acceptable salt, amide, ester or prodrug thereof wherein,
X is selected from OH, —O-alkyl, —SH, —S-alkyl, —NH₂, —NHR₆, —NR₆R₇;
R₁ and R₂ are independently selected from hydrogen, alkyl, alkylcarbonyl, alkoxycarbonyl, alkylsulfonyl, arylcarbonyl, arylsulfonyl, haloalkylcarbonyl, haloalkylsulfonyl, heterocyclecarbonyl, heterocyclesulfonyl, (NR₈R₉)carbonyl, (NR₈R₉)sulfonyl;
R₃, R₄ is selected from hydrogen, alkyl, aryl, cycloalkyl, haloalkyl, heterocycle;
R₅ is selected from hydrogen, alkenyl, alkenyloxyalkyl, alkoxyalkyl, alkoxycarbonyl, alkoxycarbonylalkyl, alkyl, alkylcarbonyl, alkylcarbonylalkyl, alkylcarbonyloxyalkyl, alkylsulfonylalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, arylalkylthioalkyl, arylsulfonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, heterocyclealkylthioalkyl, hydroxy, hydroxyalkyl, mercaptoalkyl, nitro, NR₁₀R₁₁, (NR₁₀R₁₁)carbonyl, (NR₁₀R₁₁)carbonylalkyl, (NR₁₀R₁₁)sulfonyl, (NR₁₀R₁₁)sulfonylalkyl;
R₆, R₇, R₈, R₉ R₁₀ and R₁₁ are each independently selected from hydrogen, alkyl, aryl, arylalkyl, cycloalkyl, cycloalkylalkyl, heterocycle, and heterocyclealkyl; and
n is between 0 and 6.

2. The compound of claim 1, wherein
X is hydroxy.

3. The compound of claim 1, wherein
X is hydroxy; and
R₃ and R₄ are haloalkyl.

4. The compound of claim 1, wherein
X is hydroxy;
R₃ and R₄ are haloalkyl; and
R₁ is alkylcarbonyl.

5. The compound of claim 4 selected from the group consisting of
N-[2-(2,2,2-Trifluoro-1-hydroxy-1-trifluoromethyl-ethyl)-naphthalen-1-yl]-acetamide;
N-[6-Hydroxy-2-(2,2,2-trifluoro-1-hydroxy-1-trifluoromethyl-ethyl)-naphthalen-1-yl]-acetamide;
N-[4-Nitro-2-(2,2,2-trifluoro-1-hydroxy-1-trifluoromethyl-ethyl)-naphthalen-1-yl]-acetamide;
8-Acetylamino-7-(2,2,2-trifluoro-1-hydroxy-1-trifluoromethyl-ethyl)-naphthalene-2-sulfonic acid;
5-Acetylamino-6-(2,2,2-trifluoro-1-hydroxy-1-trifluoromethyl-ethyl)-naphthalene-2-sulfonic acid;
4-Acetylamino-3-(2,2,2-trifluoro-1-hydroxy-1-trifluoromethyl-ethyl)-naphthalene-1-sulfonic acid;
N-[5-Hydroxy-2-(2,2,2-trifluoro-1-hydroxy-1-trifluoromethyl-ethyl)-naphthalen-1-yl]-acetamide;
N-[4-Hydroxy-2-(2,2,2-trifluoro-1-hydroxy-1-trifluoromethyl-ethyl)-naphthalen-1-yl]-acetamide;
N-[4-Chloro-2-(2,2,2-trifluoro-1-hydroxy-1-trifluoromethyl-ethyl)-naphthalen-1-yl]-acetamide;
-[4-Cyano-2-(2,2,2-trifluoro-1-hydroxy-1-trifluoromethyl-ethyl)-naphthalen-1-yl]-acetamide;
N-[2-(2,2,2-Trifluoro-1-hydroxy-1-trifluoromethyl-ethyl)-naphthalen-1-yl]-propionamide;
N-[2-(2,2,2-Trifluoro-1-hydroxy-1-trifluoromethyl-ethyl)-naphthalen-1-yl]-butyramide;
N-[2-(2,2,2-Trifluoro-1-hydroxy-1-trifluoromethyl-ethyl)-naphthalen-1-yl]-isobutyramide;
2-Methyl-N-[2-(2,2,2-trifluoro-1-hydroxy-1-trifluoromethyl-ethyl)-naphthalen-1-yl]-butyramide;
3-Methyl-N-[2-(2,2,2-trifluoro-1-hydroxy-1-trifluoromethyl-ethyl)-naphthalen-1-yl]-butyramide;
Pentanoic acid [2-(2,2,2-trifluoro-1-hydroxy-1-trifluoromethyl-ethyl)-naphthalen-1-yl]-amide;
Hexanoic acid [2-(2,2,2-trifluoro-1-hydroxy-1-trifluoromethyl-ethyl)-naphthalen-1-yl]-amide;
2-Methyl-pentanoic acid [2-(2,2,2-trifluoro-1-hydroxy-1-trifluoromethyl-ethyl)-naphthalen-1-yl]-amide;
2-Ethyl-N-[2-(2,2,2-trifluoro-1-hydroxy-1-trifluoromethyl-ethyl)-naphthalen-1-yl]-butyramide;
4-Methyl-pentanoic acid [2-(2,2,2-trifluoro-1-hydroxy-1-trifluoromethyl-ethyl)-naphthalen-1-yl]-amide;
3,3-Dimethyl-N-[2-(2,2,2-trifluoro-1-hydroxy-1-trifluoromethyl-ethyl)-naphthalen-1-yl]-butyramide; and
N-[4-Bromo-2-(2,2,2-trifluoro-1-hydroxy-1-trifluoromethyl-ethyl)-naphthalen-1-yl]-acetamide.

6. The compound of claim 1, wherein
X is hydroxy;
R₃ and R₄ are haloalkyl; and
R₁ is alkenylcarbonyl.

7. The compound of claim 6 selected from the group consisting of
But-2-enoic acid [2-(2,2,2-trifluoro-1-hydroxy-1-trifluoromethyl-ethyl)-naphthalen-1-yl]-amide;
3-Methyl-but-2-enoic acid [2-(2,2,2-trifluoro-1-hydroxy-1-trifluoromethyl-ethyl)-naphthalen-1-yl]-amide; and
Pent-4-enoic acid [2-(2,2,2-trifluoro-1-hydroxy-1-trifluoromethyl-ethyl)-naphthalen-1-yl]amide.

8. The compound of claim 1, wherein
X is hydroxy;
R₃ and R₄ are haloalkyl; and
R₁ is alkoxyalkylcarbonyl.

9. The compound of claim 8 that is 2-Methoxy-N-[2-(2,2,2-trifluoro-1-hydroxy-1-trifluoromethyl-ethyl)-naphthalen-1-yl]-acetamide.

10. The compound of claim 1, wherein
X is hydroxy;
R₃ and R₄ are haloalkyl; and
R₁ is cycloalkylalkylcarbonyl.

11. The compound of claim 10 selected from the group consisting of
Cyclopropanecarboxylic acid [2-(2,2,2-trifluoro-1-hydroxy-1-trifluoromethyl-ethyl)-naphthalen-1-yl]-amide;
Cyclobutanecarboxylic acid [2-(2,2,2-trifluoro-1-hydroxy-1-trifluoromethyl-ethyl)-naphthalen-1-yl]-amide;
Cyclopentanecarboxylic acid [2-(2,2,2-trifluoro-1-hydroxy-1-trifluoromethyl-ethyl)-naphthalen-1-yl]-amide;
2-Cyclopentyl-N-[2-(2,2,2-trifluoro-1-hydroxy-1-trifluoromethyl-ethyl)-naphthalen-1-yl]-acetamide; and
Cyclohexanecarboxylic acid [2-(2,2,2-trifluoro-1-hydroxy-1-trifluoromethyl-ethyl)-naphthalen-1-yl]-amide.

12. The compound of claim 1, wherein
X is hydroxy;
R₃ and R₄ are haloalkyl; and
R₁ is arylalkylcarbonyl.

13. The compound of claim 12 that is ³-Phenyl-N-[2-(2,2,2-trifluoro-1-hydroxy-1-trifluoromethyl-ethyl)-naphthalen-1-yl]-propionamide.

14. The compound of claim 1, wherein
X is hydroxy;
R₃ and R₄ are haloalkyl; and
R₁ is arylcarbonyl.

15. The compound of claim 13 selected from the group consisting of
N-[2-(2,2,2-Trifluoro-1-hydroxy-1-trifluoromethyl-ethyl)-naphthalen-1-yl]-benzamide;
2-Methyl-N-[2-(2,2,2-trifluoro-1-hydroxy-1-trifluoromethyl-ethyl)-naphthalen-1-yl]-benzamide;
3-Methyl-N-[2-(2,2,2-trifluoro-1-hydroxy-1-trifluoromethyl-ethyl)-naphthalen-1-yl]-benzamide;
4-Methyl-N-[2-(2,2,2-trifluoro-1-hydroxy-1-trifluoromethyl-ethyl)-naphthalen-1-yl]-benzamide;
4-Fluoro-N-[2-(2,2,2-trifluoro-1-hydroxy-1-trifluoromethyl-ethyl)-naphthalen-1-yl]-benzamide;
N-[2-(2,2,2-Trifluoro-1-hydroxy-1-trifluoromethyl-ethyl)-naphthalen-1-yl]-4-trifluoromethyl-benzamide;
4-Bromo-N-[2-(2,2,2-trifluoro-1-hydroxy-1-trifluoromethyl-ethyl)-naphthalen-1-yl]-benzamide;
Biphenyl-4-carboxylic acid [2-(2,2,2-trifluoro-1-hydroxy-1-trifluoromethyl-ethyl)-naphthalen-1-yl]-amide;
N-[2-(2,2,2-Trifluoro-1-hydroxy-1-trifluoromethyl-ethyl)-naphthalen-1-yl]-4-trifluoromethoxy-benzamide;
3-Fluoro-N-[2-(2,2,2-trifluoro-1-hydroxy-1-trifluoromethyl-ethyl)-naphthalen-1-yl]-benzamide;
3-Bromo-N-[2-(2,2,2-trifluoro-1-hydroxy-1-trifluoromethyl-ethyl)-naphthalen-1-yl]-benzamide;
N-[2-(2,2,2-Trifluoro-1-hydroxy-1-trifluoromethyl-ethyl)-naphthalen-1-yl]-3-trifluoromethyl-benzamide;
3-Methoxy-N-[2-(2,2,2-trifluoro-1-hydroxy-1-trifluoromethyl-ethyl)-naphthalen-1-yl]-benzamide;
3-Nitro-N-[2-(2,2,2-trifluoro-1-hydroxy-1-trifluoromethyl-ethyl)-naphthalen-1-yl]-benzamide;
3-Cyano-N-[2-(2,2,2-trifluoro-1-hydroxy-1-tryfluoromethyl-ethyl)-naphthalen-1-yl]-benzamide;
4-Iodo-N-[2-(2,2,2-trifluoro-1-hydroxy-1-trifluoromethyl-ethyl)-naphthalen-1-yl]-benzamide;
2,3-Dichloro-N-[2-(2,2,2-trifluoro-1-hydroxy-1-trifluoromethyl-ethyl)-naphthalen-1-yl]-benzamide;
3,4-Dichloro-N-[2-(2,2,2-trifluoro-1-hydroxy-1-trifluoromethyl-ethyl)-naphthalen-1-yl]-benzamide;
3,5-Dichloro-N-[2-(2,2,2-trifluoro-1-hydroxy-1-trifluoromethyl-ethyl)-naphthalen-1-yl]-benzamide;
4-Chloro-N-[2-(2,2,2-trifluoro-1-hydroxy-1-trifluoromethyl-ethyl)-naphthalen-1-yl]-benzamide;
3-Chloro-N-[2-(2,2,2-trifluoro-1-hydroxy-1-trifluoromethyl-ethyl)-naphthalen-1-yl]-benzamide; and
2-Chloro-N-[2-(2,2,2-trifluoro-1-hydroxy-1-trifluoromethyl-ethyl)-naphthalen-1-yl]-benzamide.

16. The compound of claim 1, wherein
X is hydroxy;
R₃ and R₄ are haloalkyl; and
R₁ is heterocyclecarbonyl.

17. The compound of claim 16 selected from the group consisting of
Isoxazole-5-carboxylic acid [2-(2,2,2-trifluoro-1-hydroxy-1-trifluoromethyl-ethyl)-naphthalen-1-yl]-amide;
N-[2-(2,2,2-Trifluoro-1-hydroxy-1-trifluoromethyl-ethyl)-naphthalen-1-yl]-nicotinamide;
Benzo[b]thiophene-2-carboxylic acid [2-(2,2,2-trifluoro-1-hydroxy-1-trifluoromethyl-ethyl)-naphthalen-1-yl]-amide;
Quinoxaline-2-carboxylic acid [2-(2,2,2-trifluoro-1-hydroxy-1-trifluoromethyl-ethyl)-naphthalen-1-yl]-amide;
Furan-2-carboxylic acid [2-(2,2,2-trifluoro-1-hydroxy-1-trifluoromethyl-ethyl)-naphthalen-1-yl]-amide; and
Thiophene-2-carboxylic acid [2-(2,2,2-trifluoro-1-hydroxy-1-trifluoromethyl-ethyl)-naphthalen-1-yl]-amide.

18. The compound of claim 1, wherein
X is hydroxy;
R₃ and R₄ are halo alkyl;
R₁ is (NR₈R₉)carbonyl; and
R₈ is aryl.

19. The compound of claim 18 that is 1-(4-Chloro-phenyl)-3-[2-(2,2,2-trifluoro-1-hydroxy-1-trifluoromethyl-ethyl)-naphthalen-1-yl]-urea.

20. The compound of claim 1, wherein
X is hydroxy;
R₃ and R₄ are haloalkyl;
R₁ is alkoxycarbonyl.

21. The compound of claim 20 that is [2-(2,2,2-Trifluoro-1-hydroxy-1-trifluoromethyl-ethyl)-naphthalen-1-yl]-carbamic acid isopropyl ester.

22. The compound of claim 1, wherein
X is hydroxy;
R₃ and R₄ are haloalkyl;
R₁ is haloalkylcarbonyl.

23. The compound of claim 1, wherein
X is hydroxy;
R₃ and R₄ are haloalkyl;
R₁ is alkylsulfonyl.

24. The compound of claim 1, wherein
X is hydroxy;
R₃ and R₄ are haloalkyl;
R₁ is (NR₈R₉)sulfonyl.

25. The compound of claim 1, wherein
X is hydroxy;
R₃ is haloalkyl;
R₄is aryl;
R₁ is alkylcarbonyl.

26. The compound of claim 1, wherein
X is hydroxy;
R₃ and R₄ are alkyl;
R₁ is alkylcarbonyl.

27. The compound of claim 1, wherein
X is O-alkyl;
R₃ and R₄ are haloalkyl;
R₁ is alkylcarbonyl.

28. The compound of claim 1, wherein
X is NH₂;
R₃ and R₄ are haloalkyl;
R₁ is alkylcarbonyl.

29. The compound of claim 1, wherein
X is NHR₅;
R₃ and R₄ are haloalkyl;
R₁ is alkylcarbonyl.

30. The compound of claim 1, wherein
X is —SH;
R₃ and R₄ are haloalkyl;
R₁ is alkylcarbonyl.

31. A pharmaceutical composition comprising a therapeutically effective amount of a compound of claim 1 in combination with a pharmaceutically acceptable carrier.

32. A method of treating disorders in mammals that are ameliorated by potassium channels comprising administering a potassium channel opener.

33. A method of treating a disorder in mammals that are ameliorated by potassium channels comprising administering a therapeutically effective amount of a compound of formula (I): 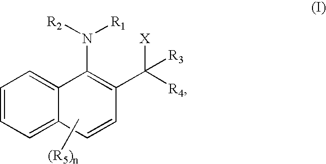
or a pharmaceutically acceptable salt thereof wherein R₁, R₂, R₃, R₄, R₅ and X are defined in claim 1.

34. A method of claim 33 wherein the disorder is selected from the group consisting of bladder overactivity, pollakiuria, bladder instability, nocturia, bladder hyperreflexia, urinary incontinence, and enuresis.

35. A method of claim 33 wherein the disorder is bladder overactivity.

36. A method of claim 33 wherein the disorder is pain.

37. A method of claim 33 wherein the disorder is BPH.

38. A method of claim 33 wherein the disorder is selected from the group consisting of male erectile dysfunction, impotence and premature ejaculation.

39. A method of claim 33 wherein the disorder is femal sexual dysfunction

40. A method of claim 33 wherein the disorder is selected from the group consisting of premature labor and dysmenorrhoea.

41. A method of claim 33 wherein the disorder is functional bowel disorder.

42. A method of claim 33 wherein the disorder is asthma.

43. A method of claim 33 wherein the disorder is epilepsy.

44. A method of claim 33 wherein the disorder is selected from the group consisting of cerebral ischemia, stroke, Alzheimer's disease and Parkinson's disease.

45. A method of claim 33 wherein the disorder is Raynaud's syndrome.

46. A method of claim 33 wherein the disorder is obesity.

47. A method of claim 33 wherein the disorder is hair loss.

48. A method of claim 33 wherein the disorder is heart diseases.

49. A method of claim 33 wherein the disorder is coronary artery disease.
